(19) United States
(12) Patent Application Publication
(10) Pub. No.: US 2025/0352339 A1
(43) Pub. Date: Nov. 20, 2025

(54) DELIVERY ASSEMBLIES WITH INFLATABLE BALLOONS AXIALLY MOVABLE RELATIVE TO EACH OTHER

(71) Applicant: EDWARDS LIFESCIENCES CORPORATION, Irvine, CA (US)

(72) Inventors: SEAN CHOW, Irvine, CA (US); Eitan Atias, Tel Aviv (IL); Noa Axelrod Manela, Kadima (IL); Tri D. Tran, Fountain Valley, CA (US); Ronaldo C. Cayabyab, Mission Viejo, CA (US)

(73) Assignee: EDWARDS LIFESCIENCES CORPORATION, Irvine, CA (US)

(21) Appl. No.: 19/286,104

(22) Filed: Jul. 30, 2025

Related U.S. Application Data
(63) Continuation of application No. PCT/US2024/016429, filed on Feb. 20, 2024.
(60) Provisional application No. 63/447,457, filed on Feb. 22, 2023.

(51) Int. Cl. A61F 2/24; A61M 25/10

(52) U.S. Cl. CPC A61F 2/2433; A61M 25/1011; A61M 2025/1013

(57) ABSTRACT

The present disclosure relates to delivery assemblies for implantation of a prosthetic valve within a valvular structure in which an opening was formed in a host leaflet. In an example, the delivery assembly comprises a guest prosthetic valve and a delivery apparatus that includes an outer balloon, and an inner balloon axially movable relative to the outer balloon. The delivery apparatus can be advanced towards the host leaflet structure with the inner balloon kept in a concealed position. The inner balloon can then be advanced relative to the outer balloon, so as to be positioned inside the host leaflet, and inflated to form a leaflet opening within the host leaflet. The inner balloon can then be deflated and the outer balloon can be advanced into the leaflet opening, and inflated with the guest prosthetic valve disposed thereover, so as to expand the guest prosthetic valve inside the leaflet opening.

### CROSS-REFERENCE TO RELATED APPLICATIONS

This application is a continuation of International Application No. PCT/US2024/016429, filed Feb. 20, 2024, which claims the benefit of U.S. Provisional Application No. 63/447,457, filed Feb. 22, 2023, which is incorporated by reference herein.

### FIELD

The present disclosure relates to delivery assemblies for implantation of prosthetic valves, and to methods and devices for modifying existing valvular structures (for example, leaflets of a native heart valve or previously-implanted prosthetic valve) prior to implantation of a guest prosthetic heart valve.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches, such as transcatheter aortic valve replacement (TAVR), are used in various procedures to deliver prosthetic medical devices to locations inside the body that are not readily accessible by surgery or where access without surgery is desirable.

Transcatheter aortic valve replacement (TAVR) is one example of a minimally-invasive surgical procedure used to replace a native aortic valve. In one specific example of the procedure, an expandable prosthetic heart valve is mounted in a crimped state on the distal end of a delivery apparatus and advanced through the patient's vasculature (for example, through a femoral artery and the aorta) to the heart. The prosthetic heart valve is positioned within the native valve and expanded to its functional size.

A variant of TAVR is valve-in-valve (ViV) TAVR, where a new prosthetic heart valve replaces a previously implanted prosthetic valve. In one specific example of the procedure, a new expandable prosthetic heart valve (“guest valve”) is delivered to the heart in a crimped state, as described above for the “native” TAVR. The guest valve is positioned within the previously implanted prosthetic valve (“host valve”) and then expanded to its functional size. The host valve in a ViV TAVR procedure can be a surgically implanted prosthetic valve or a transcatheter prosthetic valve. The term “host valve” is also used herein to refer to the native aortic valve in a native TAVR procedure.

### SUMMARY

One known technique for mitigating the risk of coronary ostial obstruction involves lacerating or severing a portion of one or more leaflets of the host valve (which can be an aortic bioprosthetic valve or a native aortic valve). Lacerating or severing a portion of the leaflet(s) reduces the risk of blocking the coronary ostia when the guest prosthetic valve is implanted and displaces the leaflets of the host valve toward the inner wall of the aortic root. However, method that rely on lacerating existing leaflets, require high spatial precision and surgical skill. Moreover, once the leaflets have been lacerated, the existing heart valve may function poorly and increase the risk of aortic insufficiency, at least until a replacement prosthetic valve has been successfully implanted. If the existing leaflets have become calcified, there is a further risk that the lacerating will release particulate or other debris into the blood stream, which may make the patient susceptible to vascular occlusion or stroke.

In one of its basic configurations, a delivery assembly comprises a delivery apparatus comprising a handle and one or more shafts coupled to the handle. This basic configuration can preferably be provided with any one or more of the features described elsewhere herein, in particular with those of the examples described hereafter. However, it should be understood that the basic configuration can preferably also be provided with any one or more of the features shown in the figures and/or described in conjunction with the figures, either in addition to or alternatively to the features of the examples described hereafter.

In some examples, the delivery apparatus can comprise an outer balloon catheter extending from the handle.

In some examples, the delivery apparatus comprises an outer balloon, optionally mounted on the outer balloon catheter.

In some examples, the delivery apparatus can comprise an outer nosecone distal to the outer balloon.

In some examples, the delivery apparatus can comprise an outer nosecone shaft attached to the outer nosecone and extending through the outer balloon catheter.

In some examples, the delivery apparatus can comprise an inner balloon catheter extending from the handle through the outer nosecone shaft lumen.

In some examples, the delivery apparatus comprises an inner balloon, optionally mounted on the inner balloon catheter.

In some examples, the outer balloon catheter can optionally define an outer balloon catheter lumen.

In some examples, the outer balloon mounted on the outer balloon catheter is optionally in fluid communication with the outer balloon catheter lumen and is configured to transition between deflated and inflated states thereof.

In some examples, the outer nosecone shaft can optionally define an outer nosecone shaft lumen.

In some examples, the inner balloon catheter can optionally define an inner balloon catheter lumen.

In some examples, the inner balloon mounted on the inner balloon catheter is optionally in fluid communication with the inner balloon catheter lumen and is optionally configured to transition between deflated and inflated states thereof.

In some examples, wherein maximum diameter of the outer balloon in its inflated state is optionally greater than the maximum diameter of the inner balloon in its inflated state.

In some examples, the inner balloon is optionally movable between a concealed position and an exposed position, wherein the inner balloon is optionally disposed proximal to a distal edge of the outer nosecone when in the concealed position, and wherein the inner balloon is optionally disposed distal to the outer nosecone when in the exposed position.

In some examples, the delivery apparatus can comprise a perforating member extending through an inner nosecone shaft lumen of an inner nosecone shaft attached to the inner nosecone.

In some examples, the perforating member is optionally configured to pierce a host leaflet of a host valvular structure to form a pilot puncture in the host leaflet, when a distal end portion of the perforating member is positioned distal to the outer nosecone and the inner nosecone.

In one of its basic methods, a method of implanting a guest prosthetic valve within a host valvular structure, comprises advancing a delivery assembly to a host valvular structure. This basic method can preferably be provided with any one or more of the steps described elsewhere herein, in particular with those of the examples described hereafter. However, it should be understood that the basic method can preferably also be provided with any one or more of the steps shown in the figures and/or described in conjunction with the figures, either in addition to or alternatively to the steps of the examples described hereafter.

In some examples, the delivery assembly optionally comprises a delivery apparatus carrying a guest prosthetic valve in a radially compressed state, to a host valvular structure.

In some examples, the delivery apparatus optionally comprises an outer balloon, and an inner balloon axially movable relative to the outer balloon.

In some examples, the method comprises positioning the inner balloon, in a deflated state thereof, within a host leaflet of the host valvular structure, by axially moving the inner balloon relative to the outer balloon.

In some examples, the method comprises inflating the inner balloon to form a leaflet opening within the host leaflet to form a leaflet opening within the host leaflet.

In some examples, the method comprises deflating the inner balloon.

In some examples, the method comprises positioning the outer balloon in a deflated state thereof, along with the guest prosthetic valve disposed in a compressed state over the outer balloon, inside the host valvular structure.

In some examples, the method comprises inflating the outer balloon, so as to radially expand the guest prosthetic valve.

In some examples, the method optionally comprises, prior to positioning the inner balloon in the host leaflet, forming, with a perforating member of the delivery apparatus, a pilot puncture within the host leaflet.

In some examples, the method positioning the inner balloon in the host leaflet optionally comprises positioning the inner balloon in the pilot puncture.

In some examples, the forming the pilot puncture optionally comprises translating the perforating member in a distal direction relative to the inner balloon and the outer balloon to pierce the host leaflet to form the pilot puncture.

In some examples, the positioning the outer balloon inside the host valvular structure optionally comprises positioning the outer balloon between host leaflets of the host valvular structure.

In some examples, the positioning the outer balloon inside the host valvular structure optionally comprises positioning the outer balloon inside the leaflet opening.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1 is a cross-sectional view of a native aortic valve.

FIG. 2A shows a cross-sectional view of a prosthetic heart valve implanted in the native aortic valve of FIG. 1, according to an example.

FIG. 2B shows the implanted prosthetic heart valve of FIG. 1A as viewed from the ascending aorta, according to an example.

FIG. 3 shows a valve-in-valve implantation within the native aortic valve of FIG. 1, according to an example.

FIG. 4 shows an exemplary delivery assembly comprising a delivery apparatus carrying a balloon expandable prosthetic device.

FIG. 5A shows a cross sectional view of an exemplary delivery apparatus comprising an inner balloon axially movable relative to an outer balloon, in a concealed position of the inner balloon.

FIG. 5B shows the delivery apparatus of FIG. 5A with an inner nosecone extending at least partially distal to an outer nosecone.

FIG. 5C shows the delivery apparatus of FIG. 5A in an exposed position of the inner balloon.

FIG. 6A shows a cross sectional view of an exemplary delivery apparatus comprising perforating member extending over a guidewire.

FIG. 6B shows the delivery apparatus of FIG. 6A with an inner nosecone extending at least partially distal to an outer nosecone.

FIG. 6C shows the delivery apparatus of FIG. 6A in an exposed position of the inner balloon.

FIG. 7A shows a distal portion of an exemplary delivery apparatus comprising an outer nosecone having a plurality of flaps, shown in a converged state of the flaps.

FIG. 7B shows the delivery apparatus of FIG. 7A, with the flaps deflected radially outwards.

FIG. 8A is a simplified side view of a delivery apparatus positioned on an outflow side of a host leaflet according to an example.

FIG. 8B is a simplified side view of the delivery apparatus of FIG. 8A with a needle piercing the host leaflet.

FIG. 8C is a simplified side view of the delivery apparatus of FIG. 8A with an inner balloon positioned within the host leaflet in a deflated state.

FIG. 8D is a simplified side view of the delivery apparatus of FIG. 8A with the inner balloon positioned within the host leaflet in an inflated state.

FIG. 9A shows the delivery apparatus of FIG. 8A with the inner balloon positioned within a pilot puncture of the host leaflet in a deflated state.

FIG. 9B shows the hole dilation balloon of FIG. 9A inflated within the host leaflet.

FIG. 10A is a simplified side view of the delivery apparatus of FIG. 8A with the inner balloon positioned within a leaflet opening in a deflated state.

FIG. 10B is a simplified side view of the delivery apparatus of FIG. 8A with a guest prosthetic valve compressed over a deflated outer balloon, positioned within the leaflet opening of the host leaflet.

FIG. 10C is a simplified side view of the delivery apparatus of FIG. 8A with the guest prosthetic valve expanded by the inflated outer balloon inside the host valvular structure.

FIG. 11 shows a previously implanted prosthetic valve subsequent to forming the leaflet opening in a host leaflet thereof.

FIG. 12 shows a configuration in which a guest prosthetic valve has been expanded within the leaflet opening of a host prosthetic valve.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a”, “an”, and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner”, “outer”, “upper”, “lower”, “inside”, “outside”, “top”, “bottom”, “interior”, “exterior”, “left”, right”, and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (for example, inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (for example, the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

The terms “axial direction”, “radial direction”, and “circumferential direction” have been used herein to describe the arrangement and assembly of components relative to the geometry of the frame of the prosthetic valve, or the geometry of an inflatable balloon that can be used to expand a prosthetic valve. Such terms have been used for convenient description, but the disclosed examples are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms “integrally formed” and “unitary” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used herein, terms such as “first”, “second”, and the like are intended to serve as respective labels of distinct components, steps, etc. and are not intended to connote or imply a specific sequence or priority. For example, unless otherwise stated, a step of performing a second action and/or of forming a second component may be performed prior to a step of performing a first action and/or of forming a first component.

As used herein, the term “substantially” means the listed value and/or property and any value and/or property that is at least 75% of the listed value and/or property. Equivalently, the term “substantially” means the listed value and/or property and any value and/or property that differs from the listed value and/or property by at most 25%. For example, “at least substantially parallel” refers to directions that are fully parallel, and to directions that diverge by up to 22.5 degrees.

In the present disclosure, a reference numeral that includes an alphabetic label (for example, “a”, “b”, “c”, etc.) is to be understood as labeling a particular example of the structure or component corresponding to the reference numeral. Accordingly, it is to be understood that components sharing like names and/or like reference numerals (for example, with different alphabetic labels or without alphabetic labels) may share any properties and/or characteristics as disclosed herein even when certain such components are not specifically described and/or addressed herein.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

Described herein are devices and methods for implanting prosthetic valves and modifying leaflets of an existing valvular structure in a patient's heart. Prior to or during implantation of the prosthetic heart valve within the existing valvular structure, each device, such as a delivery apparatus that can optionally carry a prosthetic valve, can be provided in the ascending aorta of a patient and can be used to pierce, lacerate, slice, tear, cut or otherwise modify a leaflet or commissure of the existing valvular structure. In some examples, the existing valvular structure can be a native aortic valve (for example, normal or abnormal, such as bicuspid aortic valve (BAV)) or a prosthetic valve previously implanted in the native aortic valve. The modification can avoid, or at least reduce the likelihood of, issues that leaflets of the existing valvular structure might otherwise cause once the prosthetic heart valve has been fully installed, for example, obstruction of blood flow to the coronary arteries, improper mounting due to a non-circular valve cross-section, and/or restricted access to the coronary arteries if subsequent intervention is required. While described with respect to aortic valve, it should be understood that the disclosed examples can be adapted to deliver devices that can modify existing valvular structure, and in some implementations, implant prosthetic devices, to and/or in any of the native annuluses of the heart (for example, the aortic, pulmonary, mitral, and tricuspid annuluses), and can be used with any of various delivery approaches (for example, retrograde, antegrade, transseptal, transventricular, transatrial, etc.).

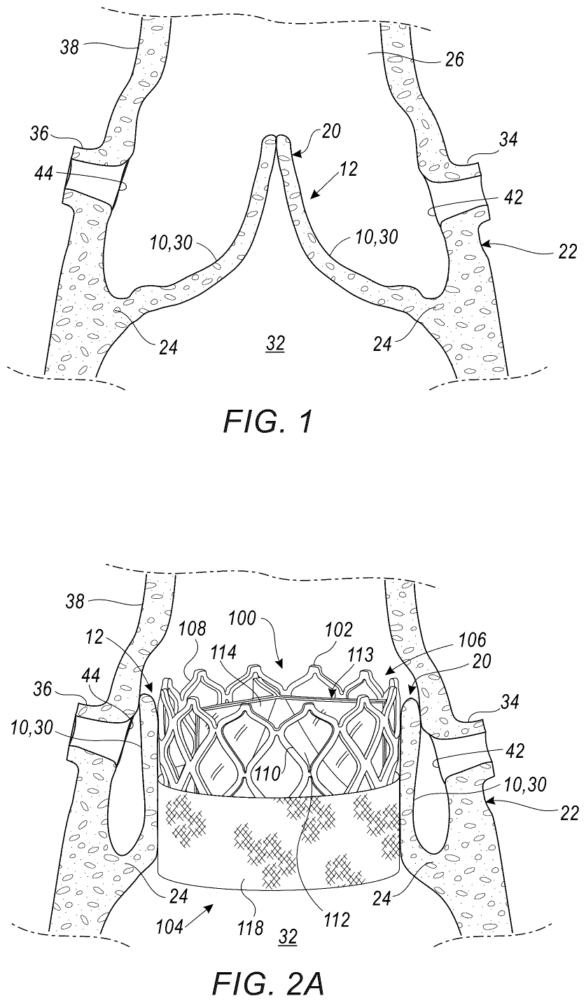

FIG. 1 illustrates an anatomy of the aortic root 22, which is positioned between the left ventricle 32 and the ascending aorta 26. The aortic root 22 includes a native aortic valve 20 having a native valvular structure 29 comprising a plurality of native leaflets 30. Normally, the native aortic valve 20 has three leaflets (only two leaflets are visible in the simplified illustration of FIG. 1), but aortic valves with fewer than three leaflets are possible. The leaflets 30 are supported at native commissures 40 (see FIG. 1B) by the aortic annulus 24, which is a ring of fibrous tissue at the transition point between the left ventricle 32 and the aortic root 22. The leaflets 30 can cycle between open and closed positions (the closed position is shown in FIG. 1) to regulate flow of blood from the left ventricle 32 to the ascending aorta 26. Branching off the aortic root 22 are the coronary arteries 34, 36. The coronary artery ostia 42, 44 are the openings that connect the aortic root 22 to the coronary arteries 34, 36.

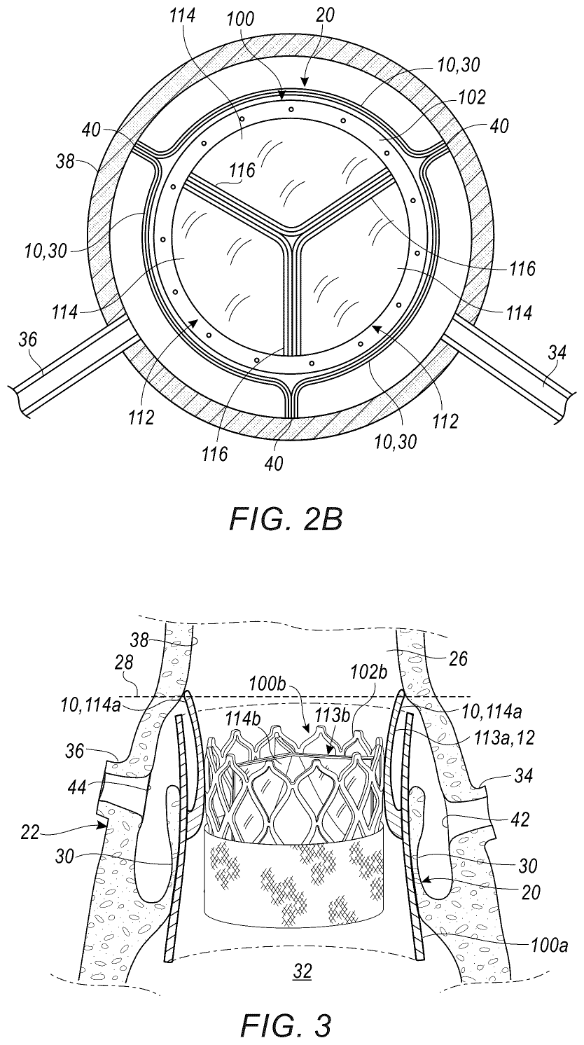

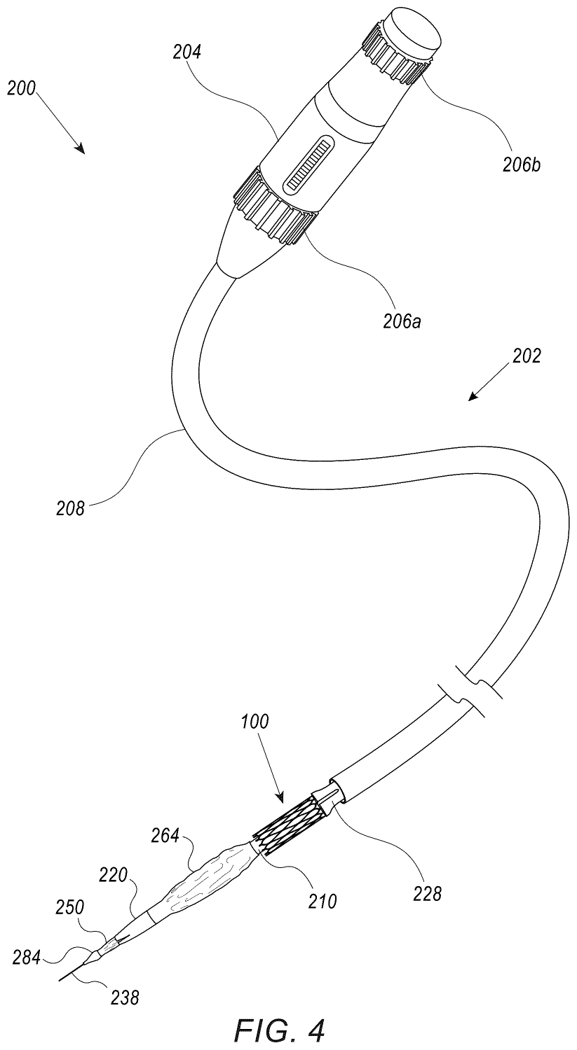

FIGS. 2A-2B show an exemplary prosthetic valve 100 that can optionally be implanted in a native heart valve, such as the native aortic valve 20 of FIG. 1. The term “prosthetic valve”, as used herein, refers to any type of a prosthetic valve deliverable to a patient's target site over a catheter, which is radially expandable and compressible between a radially compressed, or crimped, state, and a radially expanded state. Thus, the prosthetic valve can be crimped on or retained by an implant delivery apparatus (such as delivery apparatus 202 described below with respect to FIG. 4, as well as other examples of delivery apparatuses described throughout the current disclosure) in the radially compressed state during delivery, and then expanded to the radially expanded state once the prosthetic valve reaches the implantation site. The expanded state may include a range of diameters to which the valve may expand, between the compressed state and a maximum diameter reached at a fully expanded state. Thus, a plurality of partially expanded states may relate to any expansion diameter between radially compressed or crimped state, and maximum expanded state. A prosthetic valve of the current disclosure (for example, prosthetic valve 100) may include any prosthetic valve configured to be mounted within the native aortic valve, the native mitral valve, the native pulmonary valve, and the native tricuspid valve.

It is understood that the prosthetic valves disclosed herein may be used with a variety of implant delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus (not shown). Self-expandable valves include a frame that is shape-set to automatically expand as soon an outer retaining shaft or capsule (not shown) is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Pat. No. 10,603,165, International Application No. PCT/US2021/052745 and U.S. Provisional Application Nos. 63/85,947 and 63/209,904, each of which is incorporated herein by reference in its entirety), releasably coupled to respective actuation assemblies of a delivery apparatus, controlled via a handle (not shown) for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

FIGS. 2A-2B show an example of a prosthetic valve 100, which can optionally be a balloon expandable valve or any other type of valve, illustrated in an expanded state. The prosthetic valve 100 can comprise an outflow end 106 and an inflow end 104. In some instances, the outflow end 106 is the proximal end of the prosthetic valve 100, and the inflow end 104 is the distal end of the prosthetic valve 100. Alternatively, depending for example on the delivery approach of the valve, the outflow end can be the distal end of the prosthetic valve, and the inflow end can be the proximal end of the prosthetic valve.

The term “outflow”, as used herein, refers to a region of the prosthetic valve through which the blood flows through and out of the prosthetic valve 100.

The term “inflow”, as used herein, refers to a region of the prosthetic valve through which the blood flows into the prosthetic valve 100.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

In the context of the present application, the terms “lower” and “upper” are used interchangeably with the terms “distal to” and “proximal to”, respectively. Thus, for example, a lowermost component can refer to a distal-most component, and an uppermost component can similarly refer to a proximal-most component.

The terms “longitudinal” and “axial”, as used herein, refer to an axis extending in the proximal and distal directions, unless otherwise expressly defined.

The prosthetic valve 100 comprises an annular frame 102 movable between a radially compressed configuration and a radially expanded configuration, and a valvular structure 113 that comprises prosthetic valve leaflets 114 mounted within the frame 102. The frame 102 can optionally be made of various suitable materials, including plastically-deformable materials such as, but not limited to, stainless steel, a nickel based alloy (for example, a nickel-cobalt-chromium alloy such as MP35N alloy), polymers, or combinations thereof. When constructed of a plastically-deformable materials, the frame 102 can be crimped to a radially compressed state on a balloon catheter, and then expanded inside a patient by an inflatable balloon or equivalent expansion mechanism. Alternatively or additionally, the frame 102 can optionally be made of shape-memory materials such as, but not limited to, nickel titanium alloy (for example, Nitinol). When constructed of a shape-memory material, the frame 102 can be crimped to a radially compressed state and restrained in the compressed state by insertion into a shaft or equivalent mechanism of a delivery apparatus.

In the example illustrated in FIGS. 2A-2B, the frame 102 can optionally be an annular, stent-like structure comprising a plurality of intersecting struts 108. In this application, the term “strut” encompasses axial struts, angled struts, laterally extendable struts, commissure windows, commissure support struts, support posts, and any similar structures described by U.S. Pat. Nos. 7,993,394 and 9,393,110, which are incorporated herein by reference. A strut 108 may be any elongated member or portion of the frame 102. The frame 102 can include a plurality of strut rungs that can collectively define one or more rows of cells 110. The frame 102 can have a cylindrical or substantially cylindrical shape having a constant diameter from the inflow end 104 to the outflow end 106 as shown, or the frame can vary in diameter along the height of the frame, as disclosed in U.S. Pat. No. 9,155,619, which is incorporated herein by reference.

The struts 108 can optionally include a plurality of angled struts and vertical or axial struts. At least some of the struts 108 can be pivotable or bendable relative to each other, so as to permit frame expansion or compression. For example, the frame 102 can optionally be formed from a single piece of material, such as a metal tube, via various processes such as, but not limited to, laser cutting, electroforming, and/or physical vapor deposition, while retaining the ability to collapse/expand radially in the absence of hinges and like.

A valvular structure 113 of the prosthetic valve 100 can optionally include a plurality of prosthetic valve leaflets 114 (for example, three leaflets), positioned at least partially within the frame 102, and configured to regulate flow of blood through the prosthetic valve 100 from the inflow end 104 to the outflow end 106. While three leaflets 114 arranged to collapse in a tricuspid arrangement, are shown in the example illustrated in FIGS. 2A-2B, it will be clear that a prosthetic valve 100 can include any other number of leaflets 114. Adjacent leaflets 114 can optionally be arranged together to form prosthetic valve commissures 116 that are coupled (directly or indirectly) to respective portions of the frame 102, thereby securing at least a portion of the valvular structure 113 to the frame 102. The prosthetic valve leaflets 114 can optionally be made from, in whole or part, biological material (for example, pericardium), biocompatible synthetic materials, or other such materials. Further details regarding transcatheter prosthetic valves, including the manner in which leaflets 114 can be coupled to the frame 102 of the prosthetic valve 100, can be found, for example, in U.S. Pat. Nos. 6,730,118, 7,393,360, 7,510,575, 7,993,394, 8,652,202, and 11,135,56, all of which are incorporated herein by reference in their entireties.

In some examples, the prosthetic valve 100 can optionally comprise at least one skirt or sealing member. For example, the prosthetic valve 100 can optionally include an inner skirt (not shown in FIG. 2A-2B), which can be secured to the inner surface of the frame 102. Such an inner skirt can be configured to function, for example, as a scaling member to prevent or decrease perivalvular leakage. An inner skirt can further function as an anchoring region for leaflets 114 to the frame 102, and/or function to protect the leaflets 114 against damage which may be caused by contact with the frame 102, for example during valve crimping or during working cycles of the prosthetic valve 100. An inner skirt can be disposed around and attached to the inner surface of frame 102, while the leaflets can optionally be sutured to the inner skirt along a scalloped line (not shown). An inner skirt can optionally be coupled to the frame 102 via sutures or another form of coupler.

The prosthetic valve 100 can optionally comprise, in some examples, an outer skirt 118 mounted on the outer surface of frame 102 (as shown in FIGS. 2A-2B), configured to function, for example, as a sealing member retained between the frame 102 and the surrounding tissue of the native annulus against which the prosthetic valve is mounted, or against an inner side of a previously implanted valve in the case of ViV procedures (described further below), thereby reducing risk of paravalvular leakage (PVL) past the prosthetic valve 100. The outer skirt 118 can optionally be coupled to the frame 102 via sutures or another form of coupler.

Any of the inner skirt and/or outer skirt can be made of various suitable biocompatible materials, such as, but not limited to, various synthetic materials (for example, PET) or natural tissue (for example pericardial tissue). In some cases, the inner skirt can optionally be formed of a single sheet of material that extends continuously around the inner surface of frame 102. In some cases, the outer skirt 118 can optionally be formed of a single sheet of material that extends continuously around the outer surface of frame 102.

The cells 110, defined by interconnected struts 108, define cell openings 112. While some of the cell openings 112 can be covered by the inner skirt and/or the outer skirt, at least a portion of the cell opening 112 can remain uncovered, such as cell openings 112 which are closer to the outflow end 106 of the prosthetic valve.

FIGS. 2A-2B illustrate a hypothetical coronary artery obstruction that could occur in some cases from implantation of a prosthetic valve 100 within the native aortic valve 20. In this example, the prosthetic valve 100 is the guest valve or new valve, and the native aortic valve 20 is the host valve or old valve.

During implantation of the prosthetic valve 100, the prosthetic valve 100 is positioned within a central region defined between the native leaflets 30, which are also the host leaflets 10 for the example illustrated in FIG. 2A-2B. The prosthetic valve 100 is then radially expanded against the host leaflets 10. As illustrated, the host leaflets 10 form a tube around the frame 102 of the prosthetic valve 100 after the prosthetic valve 100 is radially expanded to the working diameter. As further illustrated, expansion of the prosthetic valve 100 displaces the host leaflets 10 outwards towards the coronary ostia 42, 44 such that the host leaflets 10 contact a portion of the aortic root 22 surrounding the coronary ostia 42, 44, causing coronary artery obstruction.

For an existing implanted prosthetic valve, the valvular structure may naturally degrade over time thereby requiring repair or replacement in order to maintain adequate heart functions. In a Valve-in-Valve (ViV) procedure, a new prosthetic heart valve is mounted within the existing, degrading prosthetic heart valve in order to restore proper function. FIG. 3 illustrates an exemplary hypothetical coronary artery obstruction that could occur in some cases from implantation of a prosthetic valve 100b within a previously implanted prosthetic valve 100a (for example, after a ViV procedure). In this example, the prosthetic valve 100b is the guest valve or new valve, and the prosthetic valve 100a is the host valve or old valve. In this example, the prosthetic valve 100a was previously implanted within the orifice of the native aortic valve 20. Each of the prosthetic valves 100a, 100b can have the general structure of the prosthetic valve 100 described with reference to FIGS. 2A-2B, though in some examples, each of the prosthetic valves 100a, 100b can be a different type of prosthetic valve. For example, a balloon expandable guest valve 100b can be implanted inside a previously implanted mechanically expandable or self-expandable host valve 100a.

During implantation of the prosthetic valve 100b, the prosthetic valve 100b is positioned within a central region defined between the leaflets 114a of the prosthetic valve 100a, which now take the role of host leaflet 10. The prosthetic valve 100b is then radially expanded against the host leaflets 10 (i.e., against the prosthetic valve leaflets 114c). As illustrated, the radial expansion of the prosthetic valve 100a results in outward displacement of the host leaflets 10. As further illustrated, the host leaflets 10 are displaced such that the host leaflets 10 contact the aortic root 22 at positions superior to the coronary artery ostia 42, 44, causing coronary artery ostia obstruction. Alternatively, the guest prosthetic valve 100b can displace the host leaflets 114a outwardly against the frame 102a of the host valve 100a, thereby blocking the flow of blood through the frame 102a to the coronary ostia 42, 44.

In some patient anatomies (for example, when the outflow end 106 of the prosthetic valve 100 is at the STJ level 28 and the diameter of the prosthetic valve 100 is similar to the STJ diameter such that the frame 102 touches or is very close to the aortic wall 38 at the STJ level 28), the host leaflets 10 may compromise the ability for future access into the coronary arteries 34, 36 or perfusion through the frame 102 to the coronary arteries 34, 36 during the diastole phase of the cardiac cycle. Similar problems may occur in some patient anatomies either when a guest prosthetic valve 100b is percutaneously expanded within a previously implanted host prosthetic valve 100a, or when a prosthetic valve 100 is percutaneously expanded within a native valve, displacing the native leaflets 30 outward toward the coronary ostia 42, 44.

The risk illustrated in FIG. 3 may be higher when the host valve is a bioprosthetic valve without a frame or when the leaflets of the host valve are external to a frame. Risk of coronary artery ostia obstruction can increase in a cramped aortic root or when the coronary artery ostium sits low. In the examples illustrated in FIGS. 2A-3, the host leaflets 10 are shown obstructing both coronary artery ostia 42, 44. In some cases, only one host leaflet 10 may obstruct a respective coronary artery ostium. For example, the risk of obstructing the left coronary ostium 42 tends to be greater than obstructing the right coronary ostium 44 because the left coronary ostium 42 typically sits lower than the right coronary ostium 44.

The term “host valve” as used herein refers to a native heart valve in which a prosthetic valve is implanted or a previously implanted prosthetic valve in which a new prosthetic valve is implanted. Moreover, in any of the examples disclosed herein, when the host valve is a previously implanted prosthetic valve, the host valve can optionally be a surgically implanted prosthetic heart valve (known as a “surgical valve”) or a transcatheter heart valve. The term “guest valve”, as used herein, refers to a prosthetic valve implanted in a host valve, which can optionally be cither a native heart valve or a previously implanted prosthetic valve. Similarly, the term “host leaflets 10”, as used herein, refers to native leaflets 30 of a native valve in which a new guest prosthetic valve 100 is implanted, or to prosthetic valve leaflets 114a of a previously implanted host valve 100a in which a new guest prosthetic valve 100b is implanted.

To avoid obstruction of blood flow to the coronary arteries 34, 36, the valvular structure 12 of the existing host valve (whether a native aortic valve or a previously implanted prosthetic valve) can be modified by components of a delivery apparatus prior to or during implantation of a new prosthetic valve within the existing valvular structure 12. In some examples, the host valvular structure 12 is modified by piercing, lacerating, tearing, slicing, and/or cutting one or more host leaflets 10 (for example, a free end of the host leaflet 10 or a commissure of adjacent host leaflets 10, which can be a native commissure 40 for a native aortic valve 20, or a prosthetic valve commissure 116 for a previously implanted host prosthetic valve 100) using the delivery apparatus. The modification thus disrupts the impermeable tubular structure that would otherwise be formed by the existing host leaflets 10, thereby allowing blood to flow to the coronary arteries 34, 36. Any delivery apparatus described throughout the current disclosure, is advantageously configured to modify the host valvular structure 12 (i.e., modify at least one of the host leaflets 10), and implant a guest prosthetic valve 100 within the modified valvular structure 12, without the need to switch between separate delivery apparatuses for each function.

Any delivery assembly disclosed herein, can optionally comprise a delivery apparatus according to any of the examples described below, and a balloon expandable prosthetic valve. While examples of a delivery assembly described in the current disclosure, are shown to include an exemplary delivery apparatus and a balloon expandable prosthetic valve, it should be understood that a delivery apparatus according to any example of the current disclosure can be used for implantation of other prosthetic devices aside from prosthetic valves, such as stents or grafts.

A delivery assembly comprising any delivery apparatus described throughout the current disclosure can be utilized, for example, to deliver a prosthetic aortic valve for mounting against the native aortic annulus or against a prosthetic valve previously implanted in a native aortic valve, to deliver a prosthetic mitral valve for mounting against the native mitral annulus or against a prosthetic valve previously implanted in a native mitral valve, or to deliver a prosthetic valve for mounting against any other native annulus or against a prosthetic valve previously implanted in any other native valve.

FIG. 4 illustrates an exemplary delivery assembly 200 that includes an exemplary delivery apparatus 202 adapted to deliver a balloon expandable prosthetic valve 100, such as prosthetic valve 100 described above with respect to FIGS. 2A-2B. According to some examples, the delivery apparatus 202 can optionally include a handle 204 and an outer balloon catheter 210 having an outer balloon 264 mounted on its distal end. The delivery apparatus 202 can be optionally provided with two balloons, including an inner balloon 250 axially movable relative to the outer balloon 264. The balloon expandable prosthetic valve 100 can be carried in a crimped state over the outer balloon catheter 210 or over another catheter disposed around the outer balloon catheter 210. Optionally, an outer delivery shaft 208 can concentrically extend over the outer balloon catheter 210, and a push shaft 228 can optionally be disposed over the outer balloon catheter 210, optionally between the outer balloon catheter 210 and the outer delivery shaft 208.

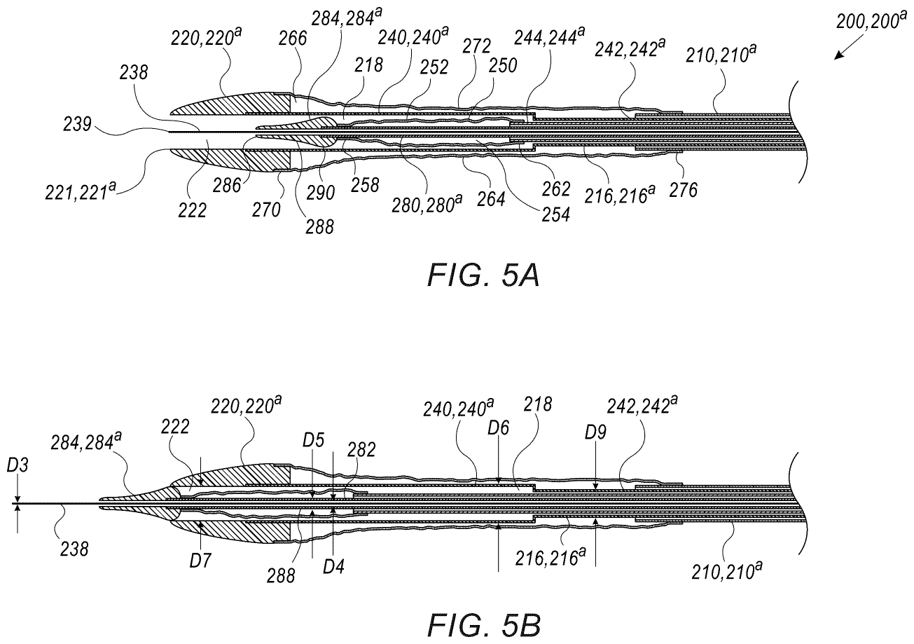

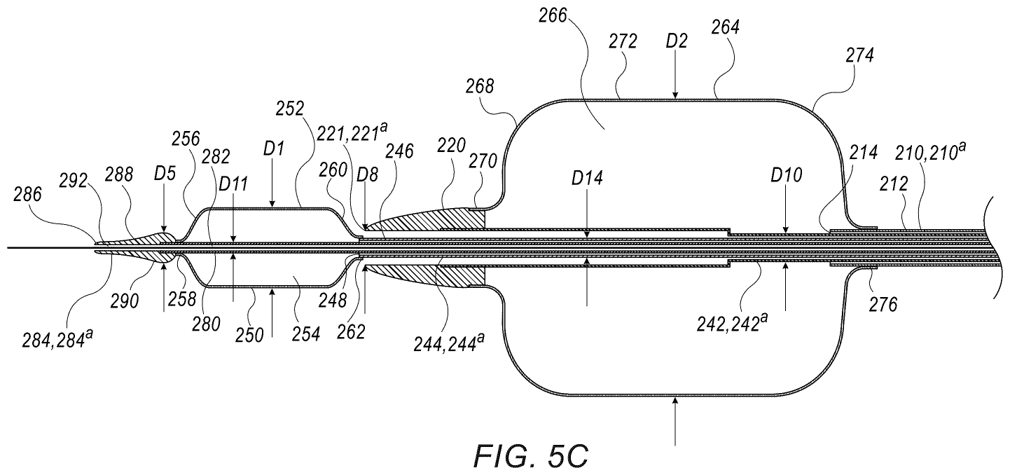

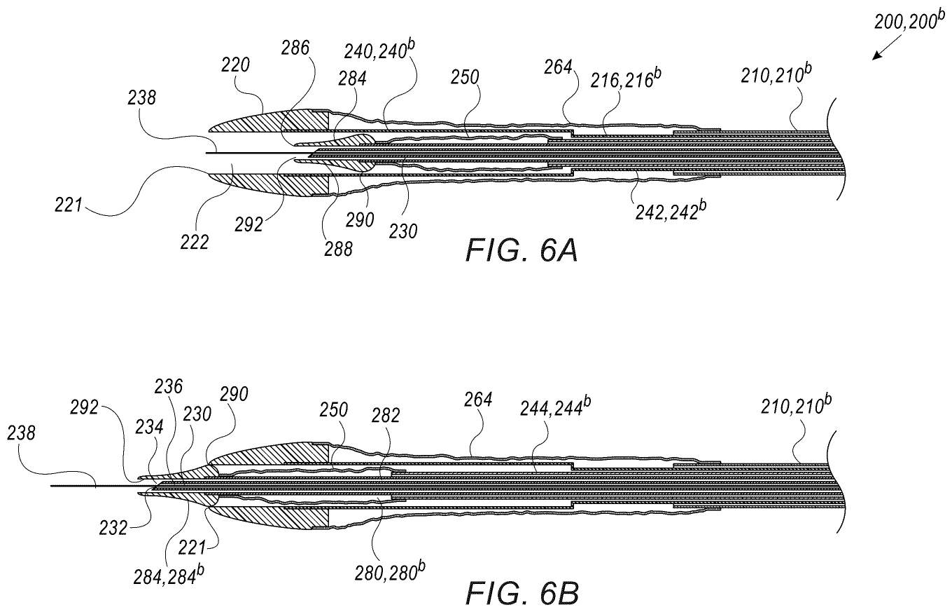

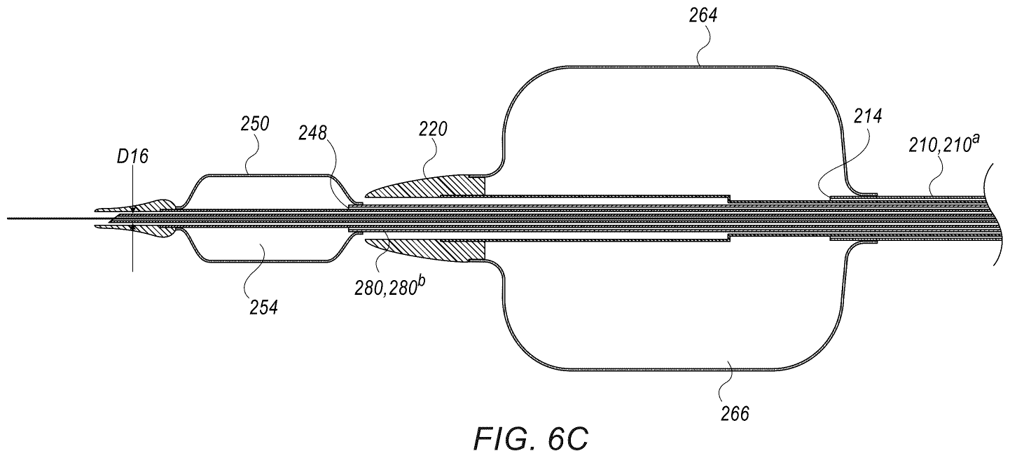

The outer delivery shaft 208, the push shaft 228, and the outer balloon catheter 210, can optionally be configured to be axially movable relative to each other. For example, a proximally oriented movement of the outer delivery shaft 208 relative to the outer balloon catheter 210, or a distally oriented movement of the outer balloon catheter 210 relative to the outer delivery shaft 208, can expose the prosthetic valve 100 from the outer delivery shaft 208. The delivery apparatus 202 further includes an outer nosecone 220 carried by an outer nosecone shaft 216 (hidden from view in FIG. 4, but shown in FIGS. 5A-6C) extending through a lumen 212 of the outer balloon catheter 210 and an internal cavity 266 of the outer balloon 264 (outer balloon catheter lumen 212 and outer balloon cavity 266 shown in FIGS. 5A-6C).

The proximal ends of the outer balloon catheter 210, the outer delivery shaft 208, the push shaft 228, and the outer nosecone shaft 216, can optionally be coupled to the handle 204. During delivery of the prosthetic valve 100, the handle 204 can be maneuvered by an operator (for example, a clinician or a surgeon) to axially advance or retract components of the delivery apparatus 202, such as the outer nosecone shaft 216, the outer balloon catheter 210, the outer delivery shaft 208, the push shaft 228, as well as additional shafts that can optionally include an inner balloon catheter 244, an inner nosecone shaft 280, and/or a perforating member 230 which will described in further detail below, through the patient's vasculature and/or along the target site of implantation, as well as to inflate the inner balloon 250 mounted on an inner balloon catheter 244, so as to enlarge a leaflet opening 52 as will be elaborated in further detail below, to inflate the outer balloon 264 mounted on the outer balloon catheter 210, so as to expand the prosthetic valve 100, and to deflate the balloon 250, 264 and retract the delivery apparatus 202 once the guest prosthetic valve 100 is mounted in the implantation site (for example, within the host valve).

The outer balloon catheter 210 can optionally extend through the handle 204 and be fluidly connectable to a fluid source for inflating the outer balloon 264. The fluid source comprises an inflation fluid. The term “inflation fluid”, as used herein, means a fluid (for example, saline, though other liquids or gas can be used) used for inflating the outer balloon 264 and/or inner balloon 250. An inflation fluid source is in fluid communication with the outer balloon catheter lumen 212, such as the annular space between the inner surface of outer balloon catheter 210 and the outer surface of outer nosecone shaft 216 extending therethrough, such that fluid from the fluid source can flow through the outer balloon catheter lumen 212, and into the outer balloon 264 to inflate it. In some examples, as will be further elaborated below, a separate inner balloon catheter 244 can optionally be also fluidly connectable to a fluid source for inflating the inner balloon 250 independently from outer balloon 264.

As will be described in greater detail below, the balloons 264, 250 are fluidly sealed from each other, wherein each of the balloons can optionally be filled by inflation fluid flowing through a different balloon catheter. For example, the outer balloon 264 can optionally be in fluid communication with the lumen 212 of the outer balloon catheter 210, such that inflation fluid provided into outer balloon catheter 210 can flow into outer balloon 264, while the inner balloon 250 can optionally be in fluid communication with the lumen 246 of the inner balloon catheter 244, such that inflation fluid provided into inner balloon catheter 244 can flow into inner balloon 250. The delivery assembly 200 can optionally further include a guidewire 238 (shown, for example, in FIGS. 5A-6C) extending through the inner balloon catheter 244, which refers to any implementation in which the guidewire 238 can pass through a lumen of any shaft extending through the inner balloon catheter 244, such as a lumen of an inner nosecone shaft and/or a lumen of a perforating member, as will be described in further detail below.

The handle 204 can optionally include a steering mechanism configured to adjust the curvature of the distal end portion of the delivery apparatus 202. In the illustrated example, the handle 204 can optionally include an adjustment member, such as the illustrated rotatable knob 206a, which in turn is operatively coupled to the proximal end portion of a pull wire. The pull wire can optionally extend distally from the handle 204 through the outer delivery shaft 208 and has a distal end portion affixed to the outer delivery shaft 208 at or near the distal end of the outer delivery shaft 208. Rotating the knob 206a can increase or decrease the tension in the pull wire, thereby adjusting the curvature of the distal end portion of the delivery apparatus 202. Further details on steering or flex mechanisms for the delivery apparatus can be found in U.S. Pat. No. 9,339,384, which is incorporated by reference herein. The handle 204 can optionally further include an adjustment mechanism including an adjustment member, such as the illustrated rotatable knob 206b. The adjustment mechanism can be configured to adjust the axial position of the push shaft 228 relative to the outer balloon catheter. The handle can optionally include additional adjustment mechanisms controllable by additional knobs to maneuver additional components of the delivery apparatus 202, such as axial movement of a perforating member 230, axial movement of an inner nosecone shaft 280, axial movement of an inner balloon catheter 244, and/or axial movement of a push shaft 228, relative to other shafts of the delivery apparatus 202, as will be elaborated in greater detail below.

The prosthetic valve 100 can optionally be carried by the delivery apparatus 202 during delivery in a crimped state, and expanded by inflation of outer balloon 264 to secure it in a native heart valve annulus (such as aortic annulus 24) or against a previously implanted prosthetic valve. In an exemplary implantation procedure, the prosthetic valve 100 can optionally be initially crimped over the outer balloon catheter 210, proximal to the outer balloon 264. Because prosthetic valve 100 is crimped at a location different from the location of outer balloon 264, prosthetic valve 100 can be crimped to a lower profile than would be possible if it was crimped on top of outer balloon 264. This lower profile permits the clinician to more easily navigate the delivery assembly 200 (including crimped prosthetic valve 100) through a patient's vasculature to the treatment location. The lower profile of the crimped prosthetic valve is helpful when navigating through portions of the patient's vasculature which are narrow, such as the iliac artery.

When reaching the host valve, the delivery apparatus 202 can be utilized to modify at least one host leaflet 10 as will be described in further detail below, after which the deflated outer balloon 264, carrying crimped prosthetic valve 100 thereover, can optionally be advanced to the target site to expand the guest prosthetic valve 100. Prior to inflation of outer balloon 264, the push shaft 228 can optionally be advanced distally, allowing its distal end portion to contact and push against the outflow end 106 of prosthetic valve 100, pushing the prosthetic valve 100 distally therewith. The distal end of push shaft 228 can be optionally dimensioned to engage with the outflow end 106 of prosthetic valve 100 in a crimped configuration of the valve. In some implementations, the distal end portion of the push shaft 228 can optionally be flared radially outward, to terminate at a wider-diameter that can contact the prosthetic valve 100 in its crimped state. Optionally, push shaft 228 can then be advanced distally, pushing the prosthetic valve 100 therewith, until the crimped prosthetic valve 100 is disposed around the outer balloon 264, after which the outer balloon 264 can optionally be inflated to radially expand the prosthetic valve 100. Once the prosthetic valve 100 is expanded to its functional diameter within a native annulus or within a previously implanted host prosthetic valve, the outer balloon 264, as well as the inner balloon 250 (if inner balloon 250 is still inflated), can optionally be deflated, and the delivery apparatus 202 can optionally be retrieved from the patient's body.

In some examples, the delivery assembly 200 can optionally be packaged in a sterile package that can be supplied to end users for storage and eventual use. In some examples, the leaflets of the prosthetic valve (typically made from bovine pericardium tissue or other natural or synthetic tissues) are treated during the manufacturing process so that they are completely or substantially dehydrated and can optionally be stored in a partially or fully crimped state without a hydrating fluid. In this manner, the package containing the delivery assembly can be free of any liquid. Methods for treating tissue leaflets for dry storage are disclosed in U.S. Pat. Nos. 8,007,992 and 8,357,387, both of which documents are incorporated herein by reference.

Each of the inner balloon 250 and outer balloon 264 is configured to transition between a deflated state and an inflated state. FIGS. 5A-6C show cross-sectional views of a distal portion of some examples of delivery assemblies 200. As shown, the inner balloon 250 is smaller in diameter than the outer balloon 264 in their fully inflated states. In some examples, the maximum diameter D1 of the inner balloon 250 in its inflated state is less than 75% of the maximum diameter D2 of the outer balloon 264 in its fully inflated state. In some examples, the maximum diameter D1 of the inner balloon 250 in its inflated state is less than 50% of the maximum diameter D2 of the outer balloon 264 in its fully inflated state. In some examples, the maximum diameter D1 of the inner balloon 250 in its inflated state is less than 40% of the maximum diameter D2 of the outer balloon 264 in its fully inflated state. In some examples, the maximum diameter D1 of inner balloon 250 in its inflated state is less than 30% of the maximum diameter D2 of the outer balloon 264 in its fully inflated state.

In some examples, the maximum diameter D1 to which the inner balloon 250 can be inflated is equal to or less than 12 mm. In some examples, the maximum diameter D1 is equal to or less than 10 mm. In some examples, the maximum diameter D1 is equal to or less than 8 mm. In some examples, the maximum diameter D2 to which the outer balloon 264 can be inflated is at least 25 mm. In some examples, the maximum diameter D2 is at least 30 mm. In some examples, the maximum diameter D2 is at least 40 mm.

As shown in FIGS. 5A-6C, the delivery apparatus 202 can optionally comprise an inner balloon catheter 244 having the inner balloon 250 mounted on its distal end. The inner balloon catheter 244 can optionally extend through the outer balloon catheter lumen 212. The delivery apparatus 202 can optionally further include an inner nosecone 284 carried by an inner nosecone shaft 280 extending through a lumen 246 of the inner balloon catheter 244.

The inner balloon 250 can optionally comprise an inner balloon distal tapering segment 256 configured to assume a tapering configuration in the inflated state of the balloon 250, extending from a narrower diameter at a distal end thereof to a wider diameter at its proximal end, and an inner balloon main segment 252 extending proximally from the inner balloon distal tapering segment 256. In some examples, the inner balloon main segment 252 can optionally have a uniform diameter along its length in the inflated state. In some examples, the inner balloon 250 can optionally further comprise an inner balloon proximal tapering segment 260 tapering from a larger diameter of the inner balloon main segment 252 to a smaller diameter at the proximal end of inner balloon proximal tapering segment 260.

The outer balloon 264 can optionally comprise an outer balloon proximal tapering segment 274 configured to assume a tapering configuration in the inflated state of the outer balloon 264, extending from a narrower diameter at a proximal end thereof to a wider diameter at its distal end, and an outer balloon main segment 272 extending distally from the outer balloon proximal tapering segment 274. In some examples, the outer balloon main segment 272 can have a uniform diameter along its length in the inflated state. In some examples, the outer balloon 264 can optionally further comprise an outer balloon distal tapering segment 268 tapering from a larger diameter of the outer balloon main segment 272 to a smaller diameter at the distal end of outer balloon distal tapering segment 268.

Inner balloon distal tapering segment 256 can optionally include an inner balloon distal attachment segment 258 that can be secured to a component of the delivery apparatus 202, such as a distal portion of inner nosecone shaft 280, a proximal portion of inner nosecone 284, or to components coupled to inner nosecone 284. Inner balloon proximal tapering segment 260 can optionally include an inner balloon proximal attachment segment 262 that can be secured to a component of the delivery apparatus 202, such as inner balloon catheter 244.

Outer balloon distal tapering segment 268 can optionally include an outer balloon distal attachment segment 270 that can be secured to a component of the delivery apparatus 202, such as a distal portion of outer balloon catheter 210, a distal portion of outer nosecone shaft 216, a proximal portion of outer nosecone 220, or to components coupled to outer nosecone 220. Outer balloon proximal tapering segment 274 can optionally include an outer balloon proximal attachment segment 276 that can be secured to a component of the delivery apparatus 202, such as outer balloon catheter 210. Any of the attachment segments 258, 262, 270, 276 can optionally be secured to another component of the delivery apparatus 202 by force fitting, heat pressing, welding or a suitable adhesive.

The outer nosecone 220 can optionally define an outer nosecone channel 222, which can be continuous with a lumen 218 of the outer nosecone shaft 216. The outer nosecone 220 can optionally taper from a narrower diameter at an outer nosecone distal edge 221 thereof to a larger diameter proximal to the outer nosecone distal edge 221. The inner nosecone 284 can optionally define an inner nosecone channel 292, which can be continuous with a lumen 282 of the inner nosecone shaft 280. The inner nosecone 284 can optionally include an inner nosecone distal tapering portion 288 that tapers from a narrower diameter of an inner nosecone distal end 286 thereof to a larger diameter of a tapering portion proximal end 290.

The outer nosecone shaft 216 can optionally be coupled, directly or indirectly, to outer nosecone 220. In some examples, a distal portion of the outer nosecone shaft 216 can optionally be coupled to a proximal portion of the outer nosecone 220, as illustrated in FIG. 5A. Attachment of the proximal portion of outer nosecone 220 to a distal portion of outer nosecone shaft 216 can be achieved by a variety of methods, such as overmolding, radio-frequency welding, through an adhesive, and/or a combination thereof. In some examples (not illustrated), the outer nosecone shaft 216 can optionally extend through the entire length of the outer nosecone channel 222, such that a distal end of the outer nosecone shaft 216 is aligned with the outer nosecone distal edge 221. In some examples (not illustrated), the outer nosecone shaft 216 can optionally be coupled to one or more components, such as collars or other connectors, which are in turn attached to the outer nosecone 220.

The inner nosecone shaft 280 can optionally be coupled, directly or indirectly, to inner nosecone 284. In some examples, a distal portion of the inner nosecone shaft 280 can optionally be coupled to a proximal portion of the inner nosecone 284, as illustrated in FIG. 5A. Attachment of the proximal portion of inner nosecone 284 to a distal portion of inner nosecone shaft 280 can be achieved by a variety of methods, such as overmolding, radio-frequency welding, through an adhesive, and/or a combination thereof. In some examples (not illustrated), the inner nosecone shaft 280 can optionally extend through the entire length of the inner nosecone channel 292, such that a distal end of the inner nosecone shaft 280 is aligned with the inner nosecone distal end 286. In some examples (not illustrated), the inner nosecone shaft 280 is coupled to one or more components, such as collars or other connectors, which are in turn attached to the inner nosecone 284.

The inner balloon catheter 244 can optionally be axially movable relative to the outer balloon catheter 210, and can optionally be configured to axially move the inner balloon 250 between a concealed position, shown in FIGS. 5A and 6A for example, in which the inner balloon 250 is retained in a deflated state, proximal to the outer nosecone distal edge 221, and a deployed position, in which the inner balloon 250 is deployed at of the outer nosecone 220, and more specifically, positioned distal to the outer nosecone distal edge 221, allowing it to be inflated to diameter D1. It is to be understood that a variety of other positions of the inner balloon, between the concealed and deployed positions, are possible, such as various partially deployed positions in which only a portion of the inner balloon 250 is disposed out of the outer nosecone 220.

FIG. 5A shows an exemplary concealed position of the inner balloon 250, in which both the inner balloon 250 and the entire inner nosecone 284 are positioned proximal to the outer nosecone distal edge 221. In some examples, the inner balloon 250 and/or inner nosecone 284 can optionally be retained within at least a portion of the outer nosecone channel 222 in the concealed position. In some examples, the inner balloon 250 and/or inner nosecone 284 can optionally be retained within at least a portion of the outer nosecone shaft lumen 218 in the concealed position.

The outer nosecone shaft 216 can optionally comprise an outer nosecone shaft distal portion 240, extending proximally from a distal end of the outer nosecone shaft 216 and/or from the proximal end of the outer nosecone 220, and an outer nosecone shaft proximal portion 242 extending from the outer nosecone shaft distal portion 240 toward the handle 204. The outer nosecone shaft distal portion 240 defines an inner diameter D6, and the outer nosecone shaft proximal portion 242 defines an inner diameter D9 and an outer diameter D10. In some examples, the outer nosecone shaft lumen 218, at least along the outer nosecone shaft distal portion 240, can optionally be sized to accommodate the inner balloon 250 and optionally the inner nosecone 284 therein, when the inner balloon 250 is in the concealed position.

Similarly, the outer nosecone shaft lumen 218 can optionally be sized to permit inner balloon catheter 244 to be passed therethrough. Inner balloon catheter 244 defines an outer diameter D14, which can optionally be, in some examples, less than the outer diameter of the inner balloon 250 in its deflated state and/or the maximum outer diameter D5 of inner nosecone 284. Thus, any portion of delivery apparatus 202 configured to accommodate and allow passage of inner nosecone 284 and/or inner balloon 250 in a deflated state thereof, such as outer nosecone channel 222 and/or outer nosecone shaft distal portion 240, can optionally also allow passage of the inner balloon catheter 244 therethrough, for example to transition the inner balloon 250 between concealed and deployed positions.

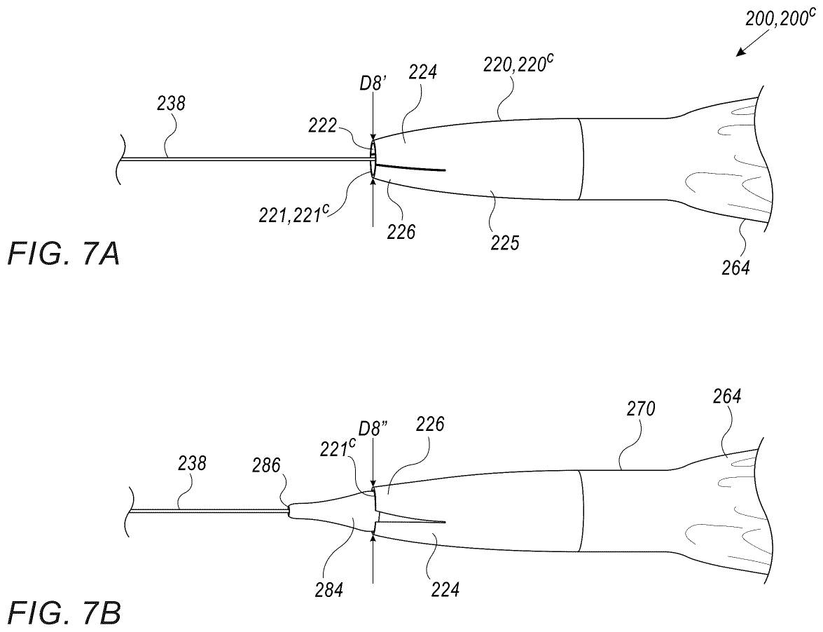

The outer nosecone channel 222 defines a diameter D7, which can optionally be, in some examples, uniform along the length of the channel 222, or can optionally be uniform along at least along a proximal portion 225 of the outer nosecone shaft (indicated in FIGS. 7A-7B, for example). Diameter D7 of outer nosecone channel 222 can optionally be dimensioned to accommodate the inner balloon 250 and optionally the inner nosecone 284 therein, and/or allow passage of the inner balloon 250 and optionally the inner nosecone 284 therethrough, when the inner balloon 250 is in the concealed position.

In some examples, the diameter D7 of outer nosecone channel 222 can optionally be substantially equal to the inner diameter D6 of outer nosecone shaft distal portion 240, together defining a continuous lumen with a uniform diameter sized to allow passage of the inner balloon 250 and optionally the inner nosecone 284 therethrough.

In some examples, the inner diameter D6 of outer nosecone shaft distal portion 240 can optionally be larger than the inner diameter D9 of the outer nosecone shaft proximal portion 242. The outer nosecone shaft 216 can optionally transition between from its distal portion 240 to its proximal portion 242 within the inner balloon cavity 254, distal to the outer balloon catheter 210, and more specifically, distal to the outer balloon catheter opening 214 at the distal end of outer balloon catheter 210. Since the outer diameter D14 of inner balloon catheter 244 can optionally be, as mentioned above, smaller than the maximum diameter D5 of the inner nosecone 284 and/or diameter D1 of the inner balloon 250 in its deflated state, the outer nosecone shaft proximal portion 242 can optionally be sized to have a relatively smaller inner diameter D9, dimensioned to allow passage of inner balloon catheter 244 but not necessarily of inner balloon 250 and/or inner nosecone 284 therethrough, which serves to provide a smaller profile of the delivery apparatus 202 proximal to the outer balloon 264.

In some examples, the inner diameter D6 of outer nosecone shaft distal portion 240 can optionally be equal to the inner diameter D9 of the outer nosecone shaft proximal portion 242, meaning that the outer nosecone shaft 216 is provided as a continuous shaft having a uniform inner diameter (example not illustrated). Such a uniform diameter of the outer nosecone shaft lumen 218 can optionally be either smaller than the diameter D7 of outer nosecone channel 222, for example allowing the inner balloon 250 (in a deflated state thereof) and/or inner nosecone 284 to pass into and out of the outer nosecone channel 222 but not necessarily the outer nosecone shaft lumen 218, or it can optionally be substantially equal to diameter D7 of outer nosecone channel 222, for example such that the inner balloon 250 (in a deflated state thereof) and/or inner nosecone 284 can pass through the entire length of the outer nosecone shaft lumen 218, including through the outer nosecone shaft proximal portion 242.

FIGS. 5B and 6B show another exemplary concealed position of the inner balloon 250, in which the inner balloon 250 is retained in a deflated state proximal to the outer nosecone distal edge 221, such as within the outer nosecone channel 222 and/or the outer nosecone shaft distal portion 240, while the inner nosecone 284 at least partially extends beyond and distally from the outer nosecone distal edge 221. For example, and as illustrated in FIGS. 5B and 6B, the inner nosecone distal tapering portion 288 can optionally extend past the outer nosecone channel 222, such that the inner nosecone distal end 286 is distal to the outer nosecone distal edge 221, while the tapering portion proximal end 290 is at, or in close proximity to, the outer nosecone distal edge 221. The outer nosecone 220 and inner nosecone 284 can optionally be shaped to define a relatively smooth transition between the nosecone at the region of the outer nosecone distal edge 221 in such a position, together defining a relatively continuous tapering outer surface formed by the distal tapering portions of both nosecone 220, 284.

The configuration shown in FIGS. 5A and 6A, as described above, can be advantageous during advancement of the delivery apparatus 202 through the patient's vasculature, taking advantage of the distal tapering portions of both nosecones 220, 284 to form a relatively elongated continuous tapering surface that facilitates delivery towards region of treatment, such as the host valvular structure 12 and host leaflet 10. Nevertheless, in some implementations, the delivery apparatus 202 can optionally be advanced through the patient vasculature while the inner nosecone 284 is concealed within the outer nosecone channel 222 and/or the outer nosecone shaft distal portion 240, as shown in FIGS. 5A and 6A for example, relying solely on the distal tapering portion of the outer nosecone 220 to facilitate delivery towards region of treatment.

FIGS. 5C and 6C show an exemplary deployed position of the inner balloon 250, in which the inner balloon as at least partially disposed out of the outer nosecone 220, and may be optionally completely exposed such that the entire inner balloon main segment 252 is positioned distal to the outer nosecone distal edge 221. This can optionally be accomplished by pushing the inner balloon catheter 244 distally relative to the outer balloon catheter 210 and/or pulling the outer balloon catheter 210 proximally relative to the inner balloon catheter 244. In this deployed position, the inner balloon 250 can optionally be inflated, optionally to the maximum diameter D1, as shown in FIGS. 5C and 6C for example.

Outer nosecone shaft 216, inner nosecone shaft 280, outer balloon catheter 210, inner balloon catheter 244, and optional outer delivery shaft 208, can optionally be formed from any of various suitable materials, such as nylon, braided stainless steel wires, or a polyether block amide (commercially available as Pebax®). In some examples, outer nosecone shaft 216, inner nosecone shaft 280, outer balloon catheter 210, inner balloon catheter 244, and optional outer delivery shaft 208, can optionally have longitudinal sections formed from different materials in order to vary the flexibility of the shafts along their lengths. In some examples, outer nosecone shaft 216 can optionally have an inner liner or layer formed of Teflon® to minimize sliding friction with any of inner nosecone 284, inner balloon 250, and/or inner balloon catheter 244.

The outer diameter D10 of outer nosecone shaft proximal portion 242 can optionally be sized such that an annular space is formed within outer balloon catheter lumen 212 between outer balloon catheter 210 and outer nosecone shaft proximal portion 242 along the length of outer balloon catheter 210. This annular space can optionally be in fluid communication with one or more outer balloon catheter openings 214 exposed to an internal cavity 266 of the outer balloon 264, which can optionally be in fluid communication with a fluid source (for example, a syringe or a pump) that can optionally inject an inflation fluid (for example, saline) into outer balloon cavity 266. In this way, fluid from the fluid source can flow through outer balloon catheter lumen 212, and into outer balloon cavity 266 via outer balloon catheter opening(s) 214, which serves to inflate the outer balloon 264 and expand and deploy a prosthetic valve 100 disposed thereon.

The outer diameter D10 of outer nosecone shaft proximal portion 242 can optionally be sized such that an annular space is formed within outer balloon catheter lumen 212 between outer balloon catheter 210 and outer nosecone shaft proximal portion 242 along the length of outer balloon catheter 210. This annular space can optionally be in fluid communication with one or more outer balloon catheter openings 214 exposed to an internal cavity 266 of the outer balloon 264, which can optionally be in fluid communication with a fluid source (for example, a syringe or a pump) that can optionally inject an inflation fluid (for example, saline) into outer balloon cavity 266. In this way, fluid from the fluid source can flow through outer balloon catheter lumen 212, and into outer balloon cavity 266 via outer balloon catheter opening(s) 214, which serves to inflate the outer balloon 264 and expand and deploy a prosthetic valve 100 disposed thereon. The pressure of the inflation fluid within outer balloon 264 may provide the force that allows the main segment 272 of outer balloon 264 to expand a prosthetic valve 100 disposed thereon. Further, the outer balloon catheter lumen 212 may optionally be configured to withdraw fluid from outer balloon cavity 266 through outer balloon catheter opening(s) 214, to deflate the outer balloon 264.

The inner nosecone shaft 280 defines an outer diameter D11 that can optionally be sized such that an annular space is formed within inner balloon catheter lumen 246 between inner balloon catheter 244 and inner nosecone shaft 280 along the length of inner balloon catheter 244. This annular space can optionally be in fluid communication with one or more inner balloon catheter openings 248 exposed to an internal cavity 254 of the inner balloon 250, which can optionally be in fluid communication with a fluid source (for example, a syringe or a pump) that can optionally inject an inflation fluid (for example, saline) into inner balloon cavity 254, which serves to inflate the inner balloon 250, for example during formation of leaflet opening 52. The pressure of the inflation fluid within inner balloon 250 may provide the force that allows the main segment 252 of inner balloon 250 to dilate a leaflet opening 52 in a manner that will be further described below. Further, the inner balloon catheter lumen 246 may optionally be configured to withdraw fluid from inner balloon cavity 254 through the inner balloon catheter opening(s) 248, to deflate the inner balloon 250.

Each of the outer balloon catheter lumen 212 and the inner balloon catheter lumen 246 can optionally be separately fed by inflation fluid, for example by being fluidly coupled to separate fluid inflation sources (for example, different syringes), or to different outlets of a common fluid inflation source, controllable to independently feed each of the lumens 212, 246. Thus, inflation fluid provided through inner balloon catheter lumen 246 can flow, through inner balloon catheter opening(s) 248, into inner balloon cavity 254, so as to inflate the inner balloon 250, without affecting the outer balloon 264. Inflation fluid can optionally be similarly drawn from inner balloon cavity 254 to deflate the inner balloon 250. Likewise, inflation fluid provided through outer balloon catheter lumen 212 can flow, through outer balloon catheter opening(s) 214, into outer balloon cavity 266, so as to inflate the outer balloon 264, without affecting the inner balloon 250. Inflation fluid can optionally be similarly drawn from outer balloon cavity 266 to deflate the outer balloon 264.

While outer balloon catheter 210 is shown to terminate at a proximal end of outer balloon 264 throughout FIGS. 5A-6C, in some examples, outer balloon catheter 210 can optionally extend farther in the distal direction (examples not illustrated), through a portion or through the entire length of outer balloon cavity 266, and one or more outer balloon catheter opening(s) 214 can optionally be formed on the sidewall of outer balloon catheter 210, exposed laterally to outer balloon cavity 266. When outer balloon catheter 210 extends farther into outer balloon cavity 266, it may optionally terminate proximal to outer nosecone shaft distal portion 240. In some examples, outer balloon catheter 210 can optionally extend around at least a portion of outer nosecone shaft distal portion 240, in which case, when the outer diameter of outer nosecone shaft distal portion 240 is larger than the outer diameter of outer nosecone shaft proximal portion 242, a distal portion of the outer balloon catheter 210 can optionally have an enlarged inner diameter, sized to accommodate the outer nosecone shaft distal portion 240 (example not illustrated).

While inner balloon catheter 244 is shown to terminate at a proximal end of inner balloon 250 throughout FIGS. 5A-6C, in some examples, inner balloon catheter 244 can optionally extend farther in the distal direction (examples not illustrated), through a portion or through the entire length of inner balloon cavity 254, and one or more inner balloon catheter opening(s) 248 can optionally be formed on the sidewall of inner balloon catheter 244, exposed laterally to inner balloon cavity 254.

Various exemplary implementations for delivery assemblies 200 can be referred to, throughout the specification, with superscripts, for case of explanation of features that refer to such exemplary implementations. It is to be understood, however, that any reference to structural or functional features of any assembly, apparatus or component, without a superscript, refers to these features being commonly shared by all specific exemplary implementations that can be also indicated by superscripts. In contrast, features emphasized with respect to an exemplary implementation of any assembly, apparatus or component, including delivery apparatus 202, referred to with a superscript, may be optionally shared by some but not necessarily all other exemplary implementations. For example, delivery apparatus 202^{a} is an exemplary implementation of delivery apparatus 202, and thus includes all of the features described for delivery apparatus 202 throughout the current disclosure, except that while a delivery apparatus 202 can be generally provided with or without a perforating member 230 extending around a guidewire 238, delivery apparatus 202^{a} does not include a perforating member 230, while delivery apparatus 202h does include a perforating member 230, as will be described in further detail below.

FIGS. 5A-5C show cross sectional views of an exemplary delivery apparatus 202^{a} at different positions of the inner balloon 250. As mentioned above, delivery apparatus 202^{a} is an exemplary implementation of delivery apparatus 202, and thus includes all of the features described for delivery apparatus 202 throughout the current disclosure, except that while delivery apparatus 202 can be provided with or without a perforating member 230, delivery apparatus 202^{a} is devoid of a separate perforating member 230 extending around the guidewire 238. The inner nosecone shaft 280^{a} defines an inner diameter D4 through which a guidewire 238 having a diameter D3 can optionally pass (i.e., D4≥D3), such that the entire delivery apparatus 202 can optionally be advanced toward the treatment region over the guidewire 238.

FIGS. 6A-6C show cross sectional views of an exemplary delivery apparatus 202^{b} at different positions of the inner balloon 250. As mentioned above, delivery apparatus 202^{b} is an exemplary implementation of delivery apparatus 202, and thus includes all of the features described for delivery apparatus 202 throughout the current disclosure, except that while delivery apparatus 202 can be provided with or without a perforating member 230, delivery apparatus 202^{b} does include a separate perforating member 230. The perforating member 230 can optionally extend through the inner balloon catheter 244, such as by extending through inner balloon catheter lumen 246 and/or a lumen of a shaft extending through inner balloon catheter 244, such as through inner nosecone shaft lumen 282 and/or inner nosecone channel 292. In some examples, the perforating member 230 can be optionally axially movable relative to another component of the delivery apparatus 202^{b}, such as the inner balloon 250. In some examples, a perforating member 230 can optionally extend through inner nosecone shaft lumen 282^{b}, and be axially movable relative to inner nosecone shaft 280^{b}. In some examples, a perforating member 230 can optionally extend through inner nosecone channel 292^{b}, and be axially movable relative to inner nosecone 284ⁿ.

Inner nosecone shaft 280^{b} defines an inner diameter D4, which is the diameter of inner nosecone shaft lumen 282^{b}. Perforating member 230 defines an outer diameter D16, and may optionally be implemented as a hollow needle, through which a guidewire 238 having a guidewire diameter D3 can optionally extend. While many conventional nosecone shafts are designed to have an inner diameter that merely allows a guidewire 238 to pass therethrough, a delivery apparatus 202^{b} that further includes a perforating member 230 passable through the inner nosecone shaft lumen 282^{b}, can optionally require the inner nosecone shaft 280^{b} to be uniquely designed to have a relatively larger inner diameter D4, to allow both the perforating member 230 and the guidewire 238 extending therethrough, to freely pass and be axially movable within the inner nosecone shaft lumen 282^{b}. Thus, a perforating member 230 can optionally have perforating member lumen 236 dimensioned similarly to conventional nosecone shaft lumens, to allow for similar axial movement of a guidewire 238 therein, necessitating the inner diameter D4 of non-conventional inner nosecone shaft 280^{b} to be, in turn, greater in size, and specifically, greater than the outer diameter D16 of the perforating member 230.

FIGS. 7A-7B show enlarged views of a distal portion of delivery apparatus 202^{c}. Delivery apparatus 202^{c} is an exemplary implementation of delivery apparatus 202, and thus includes all of the features described for delivery apparatus 202 throughout the current disclosure, except that the outer nosecone 220^{c} further comprises an outer nosecone expandable distal portion 224, extending proximally from the outer nosecone distal edge 221^{c}. The outer nosecone expandable distal portion 224 can optionally include a plurality of flaps 226 configured to deflect radially outwards when an inner nosecone 284 is pushed through the outer nosecone expandable distal portion 224. The outer nosecone 220^{c} can optionally further include an outer nosecone proximal portion 225, defined as the portion extending proximally from the outer nosecone expandable distal portion 224 (such as from the flaps 226), wherein the outer nosecone proximal portion 225 is a non-expandable portion of the outer nosecone 220^{c}.

As shown in FIG. 7A, when the inner balloon 250 is in the concealed position, with the inner nosecone 284 concealed within the outer nosecone channel 222 and/or the outer nosecone shaft distal portion 240 (similar to the position shown in FIGS. 5A and 6A), the flaps 226 can optionally converge towards each other such that the diameter D7 of the outer nosecone channel 222^{c} can narrow from a larger size at the outer nosecone proximal portion 225 to a narrower value of the diameter D8′ of the outer nosecone distal edge 221. The outer diameter D5 of the tapering portion proximal end 290 of inner nosecone 284 can optionally be greater than the diameter D8′ of the outer nosecone distal edge 221^{c} in the converged state of the flaps 226 shown in FIG. 7B, such that when the inner nosecone 284 is pushed through the outer nosecone distal edge 221^{c}, either by moving the inner nosecone shaft 280 distally relative to outer nosecone 220^{c}, or by pulling outer nosecone shaft 216 proximally relative to inner nosecone 284, the inner nosecone 284 is forcing the flaps 226 to deflect radially outward, as shown in FIG. 7B for example.

In some examples, the flaps 226 can optionally be naturally biased to the converged state shown in FIG. 7A, such that after being expanded as shown in FIG. 7B, and after the inner nosecone 284 is further pushed past outer nosecone 220^{c} or retracted back relative to outer nosecone 220^{c}, such that its tapering portion proximal end 290 is no longer pressing the flaps 226 radially outwards, the flaps 226 can optionally resiliently revert to their converging state of FIG. 7A. In some examples, the flaps 226 can optionally be plastically deformable, such that they may be kept in the converging state shown in FIG. 7A during delivery, but remain in the diverging state shown in FIG. 7B even after the tapering portion proximal end 290 is no longer aligned with the outer nosecone distal edge 221.

It is to be understood that any of the exemplary implementations of delivery apparatus 202^{a} or delivery apparatus 202^{b} can optionally be combined with any example of outer nosecone 220_{c} described above with respect to FIGS. 7A-7B, meaning that a delivery apparatus that either includes a perforating member 230 or is devoid of a perforating member, can optionally include an outer nosecone 220 that does include flaps 226 or is formed as a non-expandable unit.

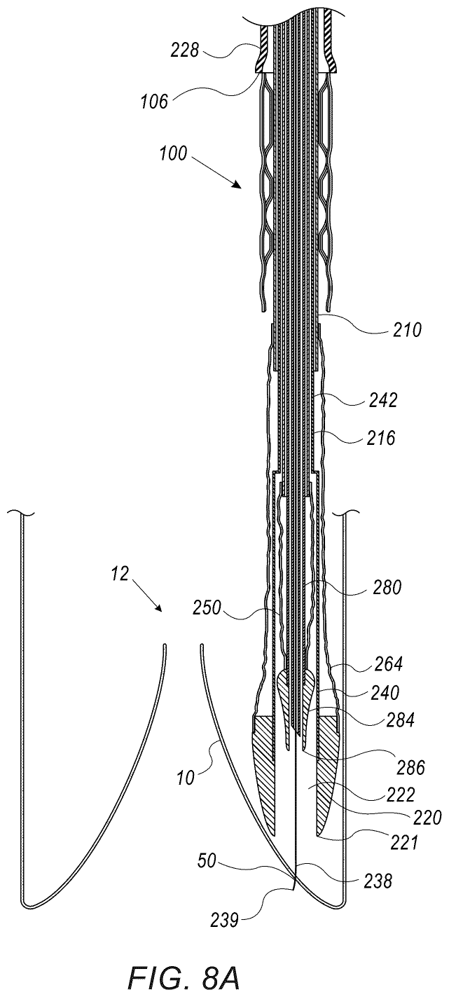

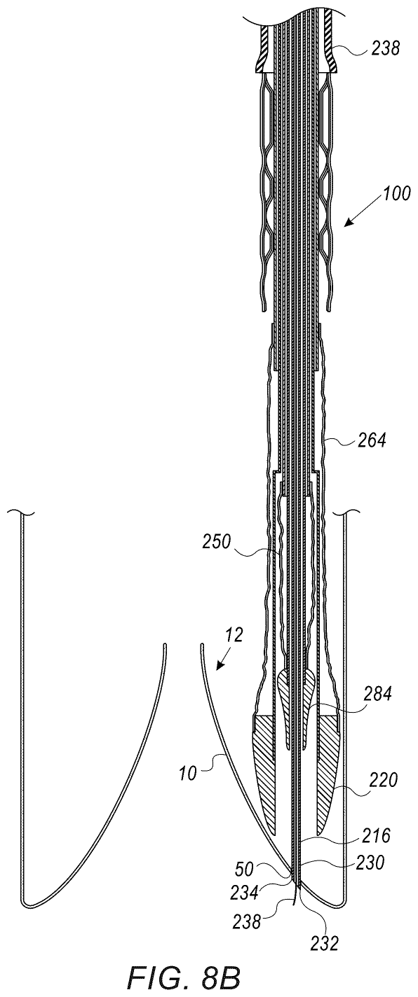

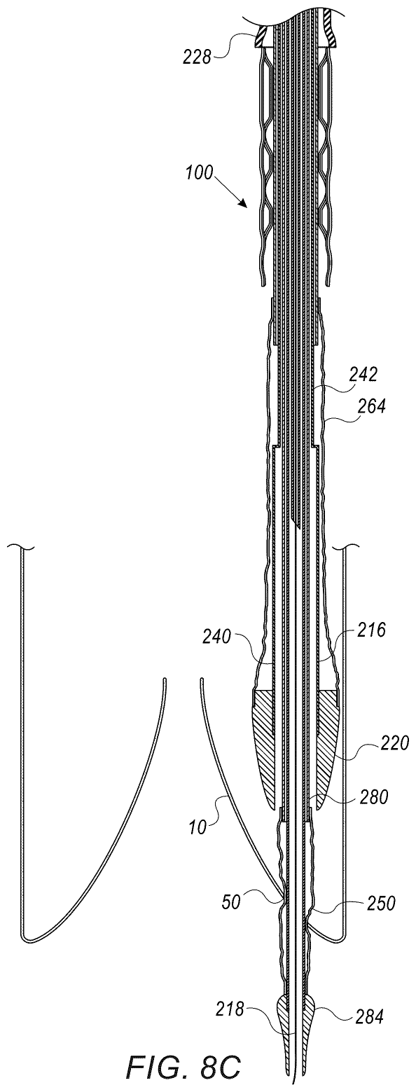

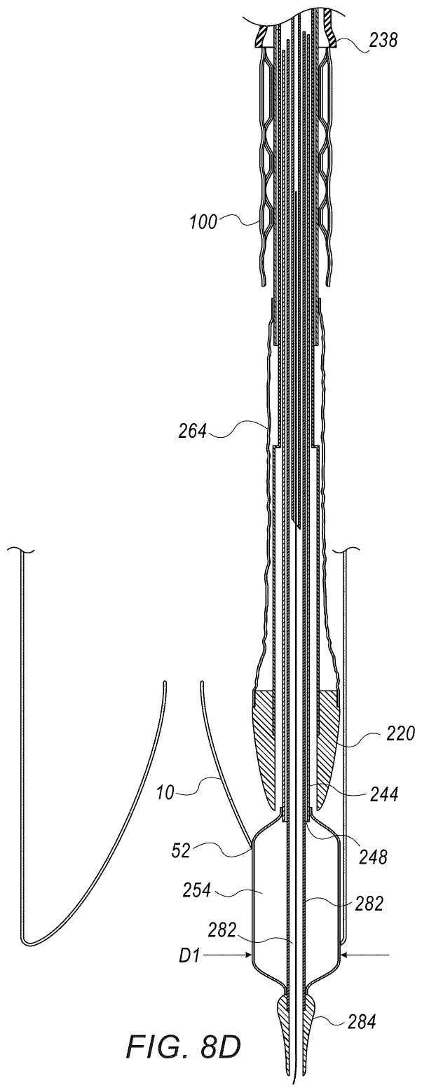

FIGS. 8A-8D illustrate some steps in a method for utilizing a delivery assembly 200 for forming a leaflet hole inside a host leaflet, which can optionally be performed prior to implanting a guest prosthetic valve inside the host valvular structure. The delivery assembly 200 can optionally be used to perforate, cut, and/or tear a host leaflet 10, such as a native leaflet 30 or a prosthetic valve leaflet 114 of a previously implanted prosthetic valve. While delivery assembly 200^{a} comprising delivery apparatus 202^{b} is illustrated throughout FIGS. 8A-8D, it is to be understood that other implementations of delivery apparatus 202 described in the current specification can be used in a similar manner, as will be further elaborated below.

The distal end portion of the delivery apparatus 202, including outer nosecone 220, is configured to be inserted into a patient's vasculature, such as optionally within an ascending aorta, and to be advanced towards the host leaflet 10, wherein the guidewire can optionally pierce through the host leaflet 10 as shown in FIG. 8A. Positioning the delivery apparatus 202 relative to the host leaflet 10 may optionally comprise advancing the delivery apparatus 202 toward the leaflet via guidewire 238. As mentioned above, the delivery apparatus 202 can optionally further include a perforating member 230, which can be also referred to as a lacerating member 230. Perforating member 230 can optionally comprise a distal end portion 232 configured to pierce a host leaflet 10 of a host valvular structure 12 to form a pilot puncture 50 in the host leaflet 10, when a distal end portion 232 is positioned distal to the inner balloon 250.

In some examples, the distal end portion 232 of perforating member 230 can optionally be configured to be selectively translated in the proximal or distal directions relative to another component of the delivery apparatus 202, such as the inner balloon 250, the outer nosecone 220 and/or the inner nosecone 284. In some examples, the inner nosecone shaft 280 and the perforating member 230 can optionally be configured to be movable axially relative to each other in the proximal and distal directions. The perforating member 230 can optionally be coupled to a handle 204. The handle 204 can optionally have one or more actuators (for example, in the form of rotatable knobs 206) that are operatively coupled to the perforating member 230 to facilitate axial movement thereof. In such examples, the distal end portion 232 can optionally be configured to pierce a host leaflet 10 when axially translated to a position which is distal to the inner balloon 250, the outer nosecone 220 and/or the inner nosecone 284. In some examples, the distal end portion 232 is not necessarily configured to be axially translatable relative to the inner balloon 250, in which case it can optionally be positioned distal to the inner balloon 250 at all times.

As shown in FIG. 8B, the perforating member 230 can optionally be configured to puncture the host leaflet 10 to form a pilot puncture 50 within host leaflet 10. The inner balloon 250 can be optionally kept in the concealed position during formation of the pilot puncture 50. Subsequent to forming the pilot puncture 50, and as shown in FIG. 8C, the inner balloon 250 may can optionally be inserted within the pilot puncture 50. This can optionally be achieved by advancing inner nosecone 284 and inner balloon 250 past the outer nosecone distal edge 221, transitioning the inner balloon 250 to the exposed position.

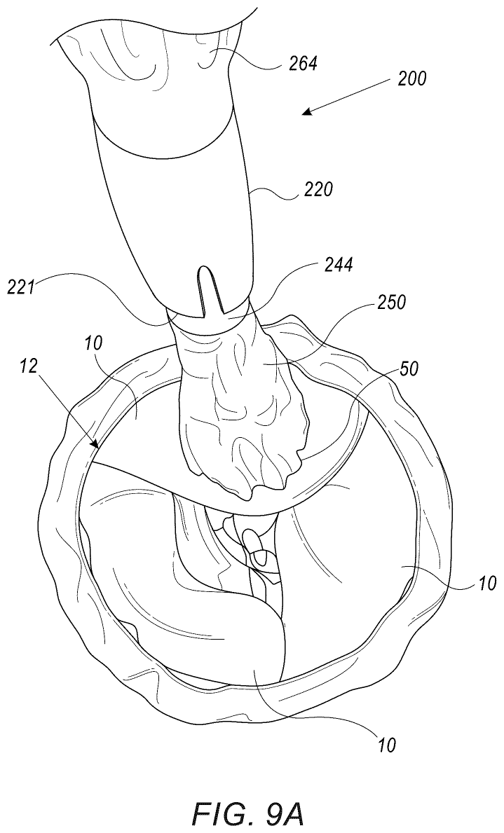

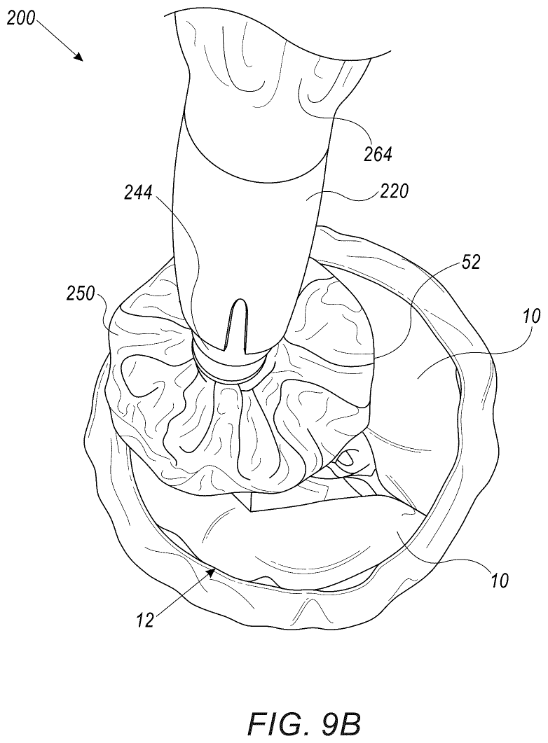

The distal end portion 232 of the perforating member 230 can optionally be concealed within a lumen of the delivery apparatus 202, being positioned proximal to the outer nosecone distal edge 221 during delivery, as shown in FIG. 9A, to avoid damage that may be caused to internal anatomical structures of the patient's body due to accidental contact with an optionally sharp distal end portion 232. The distal end portion 232 can optionally be then pushed toward and through the host leaflet 10 to form the pilot puncture 50 as shown in FIG. 8B, after which it can optionally be retracted back to re-conceal the distal end portion 232.

In some examples, the perforating member 230 can optionally be retracted so as to position the distal end portion 232 proximal to the outer nosecone distal edge 221 after forming the pilot puncture 50 and prior to advancing the inner nosecone 284 to place inner balloon 250 inside the pilot puncture 50, relying on the inner nosecone distal end 286 being small enough to allow insertion of the inner nosecone distal tapering portion 288 through pilot puncture 50. In some examples, the distal end portion 232 of the perforating member 230 can optionally remain distal to the inner nosecone distal end 286 during advancement of the inner nosecone 284 and inner balloon 250 through the pilot puncture 50, after which it is retracted to conceal the perforating member 230, positioning its distal end portion 232 proximal to the inner nosecone distal end 286, as illustrated in FIG. 8C.

While inner nosecone 284 is shown to be kept proximal to the outer nosecone distal edge 221 in FIGS. 8A-8B, prior to advancement through the pilot puncture 50, it is to be understood that the inner nosecone 284 can optionally be at least partially extend past the outer nosecone distal edge 221, for example in a position similar to that illustrated in FIGS. 5B and 6B, prior to or during formation of the pilot puncture 50, including being so-positioned during delivery of the apparatus 202 through the patient's vasculature toward the host valvular structure 12.

While inner balloon 250 is shown to be in a concealed position in FIGS. 8A-8B, prior to advancement through the pilot puncture 50, it is to be understood that the inner balloon 250 can optionally be pushed, in the deflated state, pas the outer nosecone 220, prior to or during formation of the pilot puncture 50, including prior to advancement of the guidewire 238 through the host leaflet 10 and/or prior to or during advancement of the perforating member 230 into and through the host leaflet 10.

With the inner balloon 250 received within the pilot puncture 50, inflating the inner balloon 250 to transition it from the radially deflated state (FIG. 8C) to the radially inflated state (FIG. 8D) can expand the pilot puncture 50 to form a leaflet opening 52 that is sized to receive the prosthetic valve 100 in the radially compressed or crimped configuration.

In some examples, inflating the inner balloon 250 within the host leaflet 10 serves to increase a diameter of the pilot puncture 50 such that the resulting leaflet opening 52 is a hole with an increased diameter relative to the pilot puncture 50. In some examples in which the leaflet opening 52 is a hole, the leaflet opening 52 may be a substantially circular hole. In other examples, the leaflet opening 52 may be non-circular (for example, elliptical or asymmetric). In such examples, the diameter of the leaflet opening 52 may refer to any suitable dimension of the leaflet opening 52, such as a minimum diameter of the leaflet opening 52, a maximum diameter of the leaflet opening 52, and/or an average diameter of the leaflet opening 52.

In some examples, inflating the inner balloon 250 within the host leaflet 10 may cause the host leaflet 10 to rip and/or tear such that the leaflet opening 52 is not a bounded hole. Stated differently, in such examples, the leaflet opening 52 may be formed by a tear that extends from the pilot puncture 50 fully to the free edge of the host leaflet 10 (the coaptation edge of the leaflet).

The delivery apparatus 202 may be configured to form the leaflet opening 52 in any of a variety of host valvular structures 12. In the example of FIGS. 8A-8D, the host valvular structure 12 can optionally be the valvular structure 113 of a previously implanted prosthetic valve, such as the prosthetic valve 100a of FIG. 3. In such examples, using the delivery assembly 200 as described herein to form the leaflet opening 52 in a previously implanted prosthetic valve may be followed by steps for implanting a guest prosthetic valve 100b within the previously implanted prosthetic valve 100a (for example, via a ViV procedure).

Similarly, the host valvular structure 12 in the example of FIGS. 8A-8D can optionally be a valvular structure 29 of a native heart valve, such as the native aortic valve 20 shown in FIGS. 2A-2B. In such examples, the perforating member 230 can optionally be configured to puncture a native leaflet 30 of the native aortic valve 20. In other examples, the host valvular structure and/or the native valve may refer to another valve of a patient's heart, such as a mitral valve, a pulmonary valve, or a tricuspid valve.

In some examples, the perforating member 230 may include and/or be a needle, such as a spring-loaded needle and/or a Veress needle. As shown in FIGS. 6A-6C and 8A-8D, the distal end portion 232 of the perforating member 230 can optionally terminate at an angled surface 234. The angled surface 234 can optionally have a sharp cutting edge to facilitate piercing the host leaflet 10 when the needle is pressed against the leaflet.

Delivery apparatus 202 may include any of a variety of features to facilitate positioning the outer nosecone 220, the inner nosecone 284, and/or the perforating member 230, relative to the host leaflet 10. For example, the outer nosecone shaft 216, outer balloon catheter 210, outer delivery shaft 208, inner nosecone shaft 280, inner balloon catheter 244, and/or the perforating member 230, may optionally be pre-formed, shaped, and/or curved so as to be directed and/or angled toward the host leaflet 10 when positioned in the vicinity of the host valvular structure 12. Furthermore, one or more shafts of delivery apparatus 202, such as outer delivery shaft 208, can optionally have a steering mechanism (for example, a pull wire and a corresponding adjustment mechanism in the handle 204) to steer or adjust its distal end.

As further mentioned above, in some examples, the perforating member 230 can optionally comprise a perforating member lumen 236, configured to accommodate a guidewire 238 that can optionally extend through the perforating member lumen 236. In such examples, the guidewire 238 can optionally be inserted into the patient's vasculature, and then the perforating member 230 and/or other shafts of the delivery apparatus 202 may be advanced toward the host leaflet 10 over the guidewire 238.

In some examples, the guidewire 238 can optionally be used as a perforating or lacerating member for forming a pilot puncture 50. In such examples, the guidewire 238 can optionally be a relatively stiff wire having a distal tip 239 configured to pierce the host leaflet 10 when the guidewire 238 is pressed against the leaflet. In some examples, the guidewire 238 can optionally include a radio-frequency (RF) energy delivery tip 239 to assist with penetration through the leaflet tissue. For this purpose, a suitable RF energy device may be coupled to the guidewire 238, and the RF energy device can optionally apply the RF energy to the guidewire tip 239 to penetrate the host leaflet 10. In any examples disclosed herein wherein a guidewire is used to puncture a leaflet, the guidewire can optionally be coupled to a source of RF energy that applies RF energy to the tip of the guidewire. When the guidewire 238 is used to pierce the leaflet 10, a perforating member 230 in the form of a needle, for example, can optionally be omitted as shown in FIGS. 5A-5C, or it can optionally be used in combination with the guidewire 238 that forms an initial puncture in the leaflet 10. For example, the guidewire 238 can optionally be used to form an initial pilot puncture 50 (see FIG. 8A), after which the perforating member 230 can optionally be advanced through the host leaflet 10 to form a slightly larger pilot puncture (see FIG. 8B) for subsequent advancement of the inner balloon 250 through the host leaflet 10.

In some examples, the guidewire 238 is used as a perforating member without any additional separate perforating member, such as a needle, disposed thereover (see FIGS. 5A-5C, for example), such that the guidewire 238 can optionally be utilized as the sole component that forms the pilot puncture 50, allowing other components of the delivery apparatus, such as nosecone 220, to pass through the pilot puncture 50, in a manner similar to that illustrated in FIG. 8B, but without the needle 230.

In some examples, the guidewire 238 is used as a perforating member that can optionally be used in addition to perforating member (for example, needle) 230, such that the guidewire 238 can optionally form an initial puncture via a sharp tip 239 or an RF energy delivery tip 239, as illustrated in FIG. 8A, followed by penetration of the perforating member 230 into the leaflet 10 to form the pilot puncture 50, or a pilot puncture 50 which is greater in size than an initial puncture formed by the guidewire tip 239, as shown in FIG. 8B.

In some examples, the guidewire tip 239 is not necessarily sharp enough or otherwise configured to puncture through the host leaflet 10, in which case the guidewire 238 can optionally be utilized for advancement of the delivery apparatus 202 toward the host valvular structure 12, but terminate in proximity of the host leaflet 10 without piercing through it (for example, remaining above host leaflet 10 instead of passing through the tissue as shown in FIG. 8A), and the distal end portion 232 of perforating member 230 can optionally be then advanced toward and into the host leaflet 10, to form the pilot puncture 50 in a similar manner to that illustrated in FIG. 8B.

FIGS. 9A-9B illustrate the inner balloon 250 utilized to expand the pilot puncture 50 into the leaflet opening 52. FIG. 9A illustrates the inner balloon 250 in the deflated state within the pilot puncture 50, corresponding to the state described above with respect to FIG. 8C, while FIG. 9B illustrates the inner balloon 250 in the inflated state such that the pilot puncture 50 has enlarged into the leaflet opening 52, corresponding to the state described above with respect to FIG. 8D.

As shown throughout FIGS. 8A-9B, the outer balloon 264, positioned proximal to the inner balloon 250 in the exposed position of inner balloon 250, can optionally remain in a deflated state throughout these steps of the procedure, including during inflation of inner balloon 250 shown in FIGS. 8D and 9B. When inflation fluid flows into inner balloon cavity 254 through inner balloon catheter opening 248, it causes the inner balloon to inflate and from leaflet opening 52, as shown in FIGS. 8D and 9B.

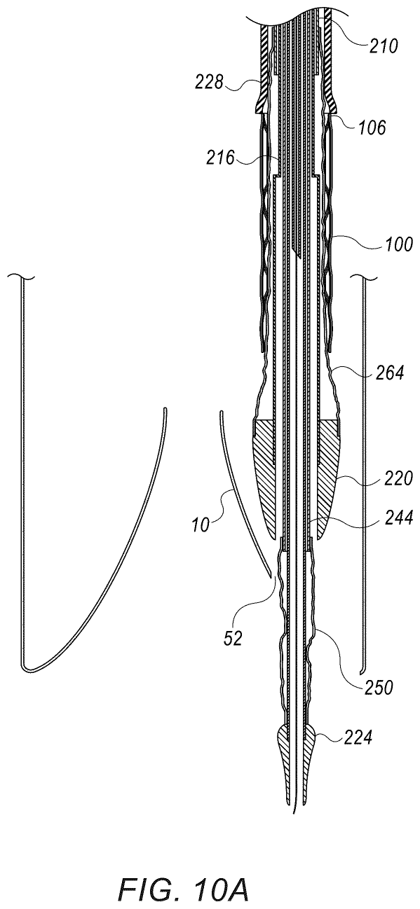

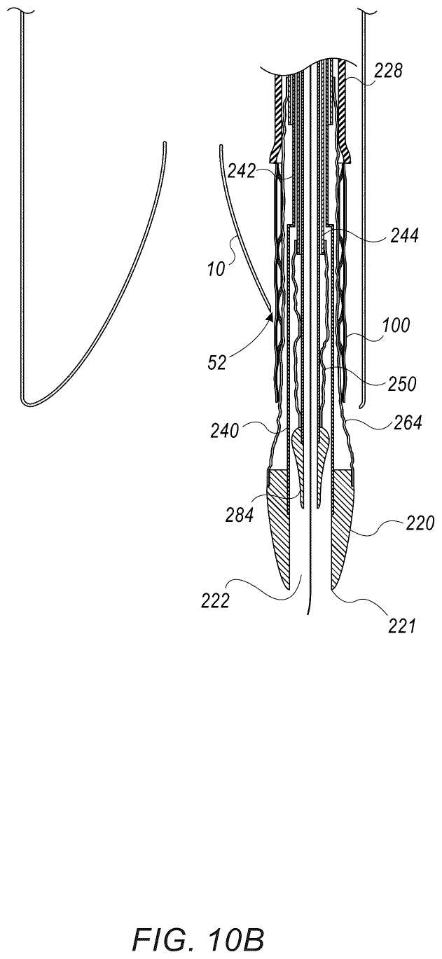

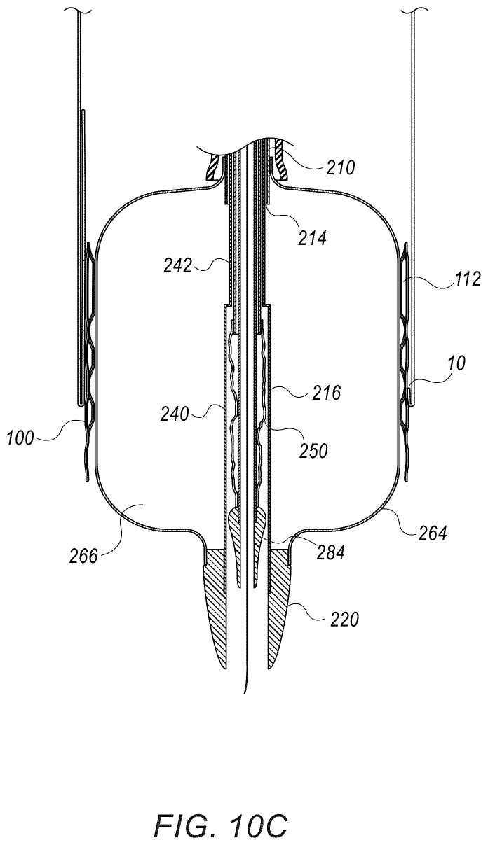

FIGS. 10A-10C show optional subsequent steps of a method utilizing delivery assembly 200, following the steps described above and illustrated in FIGS. 8A-8D. After the inner balloon 250 is inflated to form the leaflet opening 52 as shown in FIG. 8D, the inner balloon 250 can be optionally deflated, as shown in FIG. 10A, and the push shaft 228 can optionally be utilized to distally advance the crimped prosthetic valve 100 toward and around outer balloon 264.

The deflated outer balloon 264 and the prosthetic valve 100 disposed thereover can optionally be then advanced and positioned within the leaflet opening 52, as shown in FIG. 10B. The push shaft 228 can optionally remain in position, abutting outflow end 106 of prosthetic valve 100 during advancement into and through the leaflet opening 52, to provide a counter force that resists proximal displacement of the prosthetic valve 100 during insertion into leaflet opening 52.

At this stage, inflation fluid can be optionally delivered into the outer balloon cavity 266 allows the outer balloon 264 to inflate and expand the prosthetic valve 100, as shown in FIG. 10C. As described in more detail below, expanding the guest prosthetic valve 100 to the radially expanded configuration within the leaflet opening 52 of the host leaflet 10 may facilitate preserving access to the coronary arteries 34, 36 and/or maintaining sufficient perfusion of blood to the coronary arteries 34, 36 through the frame 102 of the guest prosthetic valve 100.

While the prosthetic valve 100 is shown in FIG. 10A to be pushed distally to the position of outer balloon 264 after forming the leaflet opening 52, it is to be understood that the prosthetic valve 100 can optionally be pushed by push shaft 228 over outer balloon 264 at any other stage prior to advancement of the outer balloon 264 into the leaflet opening 52 as shown in FIG. 10B. For example, the prosthetic valve 100 can optionally be pushed over outer balloon 264 upon approximation to the site of implantation, such as upon reaching the region of the ascending aorta 26 even prior to forming the pilot puncture of any of the stages shown in FIGS. 8A-8B. Similarly, the prosthetic valve 100 can optionally be pushed distally after the pilot puncture 50 is formed but before advancing the deflated inner balloon 250 into the pilot puncture 50, or after positioning the inner balloon 250 inside the pilot puncture 50 but before inflating it to form the leaflet opening 52, or after forming the leaflet opening 52 but before deflating the inner balloon 250, or after deflating the inner balloon 250. Similarly, it is to be understood that in some examples, the delivery assembly can optionally be provided without a push shaft 228, and/or that the prosthetic valve 100 can optionally be crimped around the outer balloon 264 and delivered through the patient's vasculature in this position.

With the guest prosthetic valve 100 received within the leaflet opening 52, radially expanding the guest prosthetic valve, as shown in FIG. 10C, can serve to increase a size of the leaflet opening 52 and/or to tear the leaflet. As a result, and as discussed above, radially expanding the guest prosthetic valve 100 can serve to modify the host leaflet 10 such that the leaflet does not obstruct a cell opening 112 in a frame 102 of the guest prosthetic valve 100 or at least increases the area of the host valve and the guest valve that is not covered or obstructed by the modified host leaflet to permit access and sufficient perfusion to the adjacent coronary artery. For example, radially expanding the guest prosthetic valve within the leaflet opening 52 can operate to push a portion of the leaflet extending radially exterior of the guest prosthetic valve below an upper edge of an outer skirt of the guest prosthetic valve 100 and/or away from one or more cell opening 112 of the guest prosthetic valve 100.

In some examples, the inner balloon 250 can optionally be positioned back in the concealed position, such as within outer nosecone channel 222 and/or within outer nosecone shaft distal portion 240, when the outer balloon 264 is positioned inside leaflet opening 52, as shown in FIG. 10B. In some examples, placing the inner balloon 250 back in the concealed position can optionally be achieved by advancing the outer nosecone 220 and outer balloon 264 distally into the leaflet opening 52, in a manner that covers the deflated inner balloon 250, optionally while the inner balloon 250 is kept in position, or while the inner balloon 250 is proximally pulled simultaneously with outer balloon 264 advancement.

In some examples, placing the inner balloon 250 back in the concealed position can optionally be achieved by pulling the inner balloon 250, in its deflated state, back into the outer nosecone channel 222 and/or the outer nosecone shaft distal portion 240, prior to insertion of the outer balloon 264 into the leaflet opening 52 (i.e., while the outer balloon 264 is still above the host leaflet 10 in the illustrated example).

In some examples, the inner balloon 250 can optionally remain in an exposed position but in a deflated state, even when the outer balloon 264 is inserted into the leaflet opening 52. In such cases, the inner balloon can optionally be retracted into the concealed position prior to outer balloon inflation, after outer balloon inflation, or after outer balloon deflation prior to retrieval of the delivery apparatus 202 from the patient's body. In some examples, the inner balloon 250 can optionally remain distal to the outer nosecone 220 during retrieval of the delivery apparatus 202 from the patient's body.

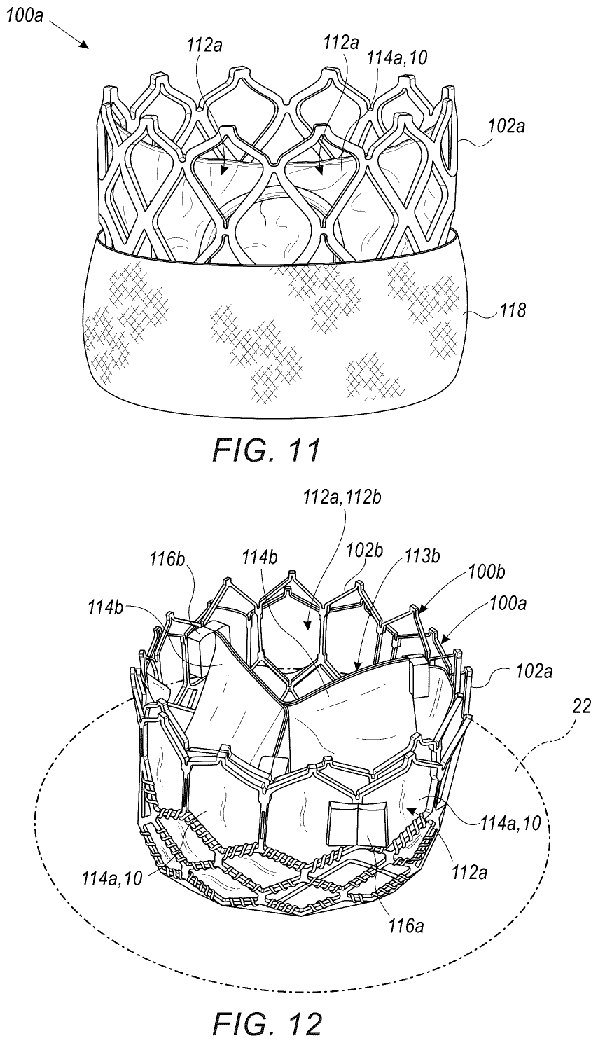

As mentioned, the delivery assemblies and methods of the current specification can be utilized for forming a leaflet opening 52 in a host leaflet 10 which can be either a native leaflet 30 or a prosthetic valve leaflet 114 of a previously implanted prosthetic valve, such as prosthetic valve 100a of FIG. 3, such as in the case of ViV procedures. FIG. 11 shows a previously implanted prosthetic valve 100a subsequent to forming the leaflet opening 52, for example subsequent to the method described above with respect to FIGS. 8A-9B. FIG. 12 shows a configuration in which a second prosthetic valve 100b has been expanded within the leaflet opening 52 of a host prosthetic valve 100a. In the example of FIG. 12, the guest prosthetic valve 100b is the same type of valve as the host prosthetic valve 100a. It is to be understood, however, that the methods described herein, when implemented in ViV procedures, also may be applied to any other suitable valvular structures, such as different prosthetic valves and/or native heart valves. For example, the guest prosthetic valve 100b need not be the same type of valve as the host prosthetic valve 100a.

In the example of FIG. 11, when the prosthetic valve leaflets 114a of the previously implanted prosthetic valve 100a are pressed against the frame 102a, the leaflet opening 52 provides a partial access into the frame 102a, but the leaflet opening 52 may not be sufficiently large to completely uncover any of the cell openings 112a of the frame 102a.

As shown in FIG. 12, however, fully expanding the guest prosthetic valve 100b within the leaflet opening 52 further expands and/or tears the leaflet opening 52 such that several cell openings 112a of the frame 102a of the host prosthetic valve 100a and several cell openings 112b of the frame 102b of the guest prosthetic valve 100b are fully uncovered by the leaflets 114a. In some examples, this may optionally result from the frame 102b of the guest prosthetic valve 100b pushing the leaflet 114a comprising the leaflet opening 52 downwardly (toward the inflow ends of the prosthetic valves 100a, 100b) such that one or more cell openings 112a are unobstructed by the leaflet 114a. In some examples, expanding the frame 102b within the leaflet 114a comprising the leaflet opening 52 may rip and/or tear this leaflet 114a such that the leaflet 114a cannot obstruct one or more cell openings 112a.

While the methods disclosed herein refer to forming a leaflet opening 52 in a host leaflet 10, prior to positioning and expanding a prosthetic valve 100, it is to be understood that any of the methods can optionally comprise, in some examples, repeating one or more steps disclosed throughout the current specification to form a plurality of punctures and openings in the host valvular structure. For example, steps described above with respect to FIGS. 8A-9B and equivalents thereof, can optionally be performed for forming a first leaflet opening in a first host leaflet, after which the hole dilation balloon can optionally be deflated, for example in a similar manner to that illustrated in FIG. 10A, and the delivery apparatus can optionally be retracted from the first host leaflet and steered toward another host leaflet, after which the same steps can optionally be repeated to form a second leaflet opening within the second host leaflet. The procedure can be optionally repeated to form further leaflet openings, such as a third leaflet opening in a third host leaflet.

In some examples, forming more than one leaflet opening, such as forming the second leaflet opening, can provide further access and/or fluid paths through the frame of the guest prosthetic valve. For example, radially expanding the guest prosthetic valve 100 within the first leaflet opening may push the second host leaflet against the frame of the guest prosthetic valve such that the second leaflet opening is aligned with cell opening(s) of the frame of the guest prosthetic valve. Thus, the second leaflet opening can provide additional unobstructed paths through the frame of the guest prosthetic valve. Moreover, in an example in which the host valve is a previously implanted prosthetic valve, expanding the guest prosthetic valve within the first leaflet opening can trap the second leaflet opening between the respective frames of the host prosthetic valve and the guest prosthetic valve, thereby providing additional access and/or flow paths through each of the frames.

Thus, forming the second leaflet opening can ensure that a greater number of cell openings of the frame are uncovered, and/or that a greater proportion of the frame is uncovered, relative to an example in which only one leaflet is punctured to form a leaflet opening. This may be particularly beneficial in examples in which the frame of a host prosthetic valve extends axially in a downstream direction beyond one or both of the coronary arteries when the guest prosthetic valve is implanted within a native heart valve.

Specifically, in some patient anatomies, the left coronary artery is positioned lower (that is, proximate to the host valvular structure) than the right coronary artery. In such examples, the right coronary artery may be sufficiently far from the host valvular structure that implanting the guest prosthetic heart valve within the host valvular structure does not limit access and/or perfusion to the right coronary artery. Accordingly, forming a single leaflet opening in the host valvular structure may be sufficient to ensure access and/or perfusion to both coronary arteries, provided that the leaflet opening is formed and/or positioned to ensure access to the left coronary artery.

In other examples, however, each of the left and right coronary arteries may be positioned sufficiently proximate to the host valvular structure that forming a single leaflet opening in the host valvular structure is insufficient to ensure access to both coronary arteries. In such examples, forming two leaflet openings in respective leaflets of the previously implanted prosthetic heart valve may ensure the ability for future access into both coronary arteries or perfusion through the frame to both coronary arteries during the diastole phase of the cardiac cycle. In some examples, the host valvular structure can optionally be modified such that the guest prosthetic valve is implanted by being expanded in a leaflet opening of a first host leaflet that faces the left coronary artery, and such that the second leaflet opening is formed in a second host leaflet that faces the right coronary artery (or vice-versa).

In some examples, forming the first leaflet opening can optionally be performed prior to forming the second leaflet opening. In other examples, forming the second leaflet opening can optionally be performed prior to forming the first leaflet opening. In some examples, the order of forming leaflet openings is chosen such that the final leaflet opening is formed in the host leaflet in which the prosthetic valve 100 is to be positioned and expanded, such as over a valve expansion balloon as described above with respect to FIGS. 11A-12C and equivalents thereof.

It is to be understood that the guest prosthetic valve 100 is not limited to being implanted within an opening 52 of a leaflet. For example, in cases where the inner balloon 250 forms a full tear in a host leaflet that extends to the coaptation edge of the leaflet, the guest prosthetic valve 100 can optionally be positioned at a location between the leaflets of the host valvular structure, for example by retracting the delivery apparatus from the host leaflet in which a leaflet opening is formed, repositioning and readvancing it such that the deflated valve expansion balloon, along with the prosthetic valve 100 disposed thereon, is positioned between the host leaflets, and then inflating the valve expansion balloon to expand the prosthetic valve 100. In some examples, such as in cases where the opening 52 does not form a full tear in the leaflet, the guest prosthetic valve can optionally be positioned at a location between the leaflets of the host valvular structure 12 (such that the delivery assembly 200 used to implant to guest prosthetic valve 100 does not extend through the leaflet opening 52) and then expanded. In such cases, the opening 52 may provide sufficient open space through which blood may flow into the coronary ostia, and/or through which additional access devices, such as coronary catheters, can pass during future interventional procedures.

Any of the assemblies, devices, apparatuses, etc. herein can optionally be sterilized (for example, with heat, radiation, and/or chemicals, etc.) to ensure they are safe for use with patients, and any of the methods herein can optionally include sterilization of the associated assembly, device, apparatus, etc. as one of the steps of the method. Examples of radiation for use in sterilization include, without limitation, gamma radiation and ultra-violet radiation. Examples of chemicals for use in sterilization include, without limitation, ethylene oxide and hydrogen peroxide.

### SOME EXAMPLES OF THE DISCLOSED IMPLEMENTATIONS

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. A delivery assembly comprising: a delivery apparatus comprising: a handle; an outer balloon catheter extending from the handle, the outer balloon catheter defining an outer balloon catheter lumen; an outer balloon mounted on the outer balloon catheter and in fluid communication with the outer balloon catheter lumen, the outer balloon configured to transition between deflated and inflated states thereof; an outer nosecone distal to the outer balloon; an outer nosecone shaft attached to the outer nosecone and extending through the outer balloon catheter, the outer nosecone shaft defining an outer nosecone shaft lumen; an inner balloon catheter extending from the handle through the outer nosecone shaft lumen, the inner balloon catheter defining an inner balloon catheter lumen; and an inner balloon mounted on the inner balloon catheter and in fluid communication with the inner balloon catheter lumen, the inner balloon configured to transition between deflated and inflated states thereof.

Example 2. The delivery assembly of any example herein, particularly example 1, wherein the maximum diameter of the outer balloon in its inflated state is greater than the maximum diameter of the inner balloon in its inflated state.

Example 3. The delivery assembly of any example herein, particularly example 1 or 2, wherein the inner balloon is movable between a concealed position and an exposed position, wherein the inner balloon is disposed proximal to a distal edge of the outer nosecone when in the concealed position, and wherein the inner balloon is disposed distal to the outer nosecone when in the exposed position.

Example 4. The delivery assembly of any example herein, particularly example 3, wherein the balloon is in its deflated state when in the concealed position.

Example 5. The delivery assembly of any example herein, particularly any one of examples 1 to 4, further comprising a guest prosthetic valve comprising a frame movable between a radially compressed and a radially expanded configuration.

Example 6. The delivery assembly of any example herein, particularly example 5, wherein, when the guest prosthetic valve is disposed around the outer balloon and positioned within a host valvular structure, inflation of the outer balloon expands the guest prosthetic valve to implant the guest prosthetic valve in the host valvular structure.

Example 7. The delivery assembly of any example herein, particularly any one of examples 1 to 6, further comprising an inner nosecone distal to the inner balloon.

Example 8. The delivery assembly of any example herein, particularly example 7, further comprising an inner nosecone shaft attached to the inner nosecone and extending through the inner balloon catheter, the inner nosecone shaft defining an inner nosecone shaft lumen.

Example 9. The delivery assembly of any example herein, particularly example 8, further comprising a perforating member extending through the inner nosecone shaft lumen, the perforating member configured to pierce a host leaflet of a host valvular structure to form a pilot puncture in the host leaflet, when a distal end portion of the perforating member is positioned distal to the outer nosecone and the inner nosecone.

Example 10. The delivery assembly of any example herein, particularly example 9, wherein the inner balloon is configured to be inserted within the pilot puncture.

Example 11. The delivery assembly of any example herein, particularly example 10, wherein inflation of the inner balloon, when positioned within the pilot puncture, is configured to expand the pilot puncture to form a leaflet opening.

Example 12. The delivery assembly of any example herein, particularly any one of examples 9 to 11, wherein the distal end portion of the perforating member is axially movable relative to the inner balloon.

Example 13. The delivery assembly of any example herein, particularly any one of examples 9 to 12, wherein the distal end portion of the perforating member is axially movable relative to the inner balloon.

Example 14. The delivery assembly of any example herein, particularly any one of examples 9 to 13, wherein the distal end portion of the perforating member is axially movable relative to the outer nosecone.

Example 15. The delivery assembly of any example herein, particularly any one of examples 9 to 14, wherein the distal end portion of the perforating member is axially movable relative to the outer nosecone shaft.

Example 16. The delivery assembly of any example herein, particularly any one of examples 9 to 15, wherein the distal end portion of the perforating member terminates at an angled surface.

Example 17. The delivery assembly of any example herein, particularly any one of examples 9 to 16, wherein the perforating member comprises a needle.

Example 18. The delivery assembly of any example herein, particularly any one of examples 9 to 17, wherein the needle is one or both of a spring-loaded needle and a Veress needle.

Example 19. The delivery assembly of any example herein, particularly any one of examples 9 to 18, wherein the perforating member comprises a perforating member lumen.

Example 20. The delivery assembly of any example herein, particularly example 19, further comprising a guidewire extending through the perforating member lumen.

Example 21. The delivery assembly of any example herein, particularly example 20, wherein the guidewire comprises a sharp tip configured to penetrate through the host leaflet.

Example 22. The delivery assembly of any example herein, particularly example 20, further comprising an RF energy source coupled to the guidewire and configured to provide RF energy to a tip of the guidewire.

Example 23. The delivery assembly of any example herein, particularly any one of examples 9 to 14, wherein the perforating member is a guidewire extending through the inner balloon catheter.

Example 24. The delivery assembly of any example herein, particularly example 23, wherein the guidewire comprises a sharp tip configured to penetrate through the host leaflet.

Example 25. The delivery assembly of any example herein, particularly example 23, further comprising an RF energy source coupled to the guidewire and configured to provide RF energy to a tip of the guidewire.

Example 26. The delivery assembly of any example herein, particularly any one of examples 7 to 25, wherein the inner nosecone is axially movable relative to the outer nosecone.

Example 27. The delivery assembly of any example herein, particularly any one of examples 7 to 26, wherein the inner nosecone is axially movable relative to the outer nosecone shaft.

Example 28. The delivery assembly of any example herein, particularly any one of examples 7 to 27, wherein the inner nosecone is axially movable relative to the outer balloon.

Example 29. The delivery assembly of any example herein, particularly any one of examples 8 to 25, wherein the inner nosecone shaft is axially movable relative to the outer nosecone.

Example 30. The delivery assembly of any example herein, particularly any one of examples 8 to 25, wherein the inner nosecone shaft is axially movable relative to the outer nosecone shaft.

Example 31. The delivery assembly of any example herein, particularly any one of examples 8 to 25, wherein the inner nosecone shaft is axially movable relative to the outer balloon.

Example 32. The delivery assembly of any example herein, particularly any one of examples 8 to 25, wherein the inner nosecone shaft is axially movable relative to the outer balloon catheter.

Example 33. The delivery assembly of any example herein, particularly any one of examples 1 to 32, wherein the inner balloon is axially movable relative to the outer balloon.

Example 34. The delivery assembly of any example herein, particularly any one of examples 1 to 33, wherein the inner balloon is axially movable relative to the outer nosecone.

Example 35. The delivery assembly of any example herein, particularly any one of examples 1 to 34, wherein the inner balloon is axially movable relative to the outer nosecone shaft.

Example 36. The delivery assembly of any example herein, particularly any one of examples 1 to 35, wherein the inner balloon is axially movable relative to the outer balloon catheter.

Example 37. The delivery assembly of any example herein, particularly any one of examples 1 to 36, wherein the inner balloon catheter is axially movable relative to the outer balloon.

Example 38. The delivery assembly of any example herein, particularly any one of examples 1 to 37, wherein the inner balloon catheter is axially movable relative to the outer nosecone.

Example 39. The delivery assembly of any example herein, particularly any one of examples 1 to 38, wherein the inner balloon catheter is axially movable relative to the outer nosecone shaft.

Example 40. The delivery assembly of any example herein, particularly any one of examples 1 to 39, wherein the inner balloon catheter is axially movable relative to the outer balloon catheter.

Example 41. The delivery assembly of any example herein, particularly any one of examples 1 to 40, wherein the outer nosecone shaft comprises an outer nosecone shaft proximal portion extending through the outer balloon catheter lumen, and an outer nosecone shaft distal portion extending between the outer nosecone and the outer nosecone shaft proximal portion.

Example 42. The delivery assembly of any example herein, particularly example 41, wherein the outer nosecone shaft distal portion defines an inner diameter that is greater than an inner diameter defined by the outer nosecone shaft proximal portion.

Example 43. The delivery assembly of any example herein, particularly example 41 or 42, wherein the outer nosecone shaft distal portion is disposed within the outer balloon.

Example 44. The delivery assembly of any example herein, particularly any one of examples 3 to 4, wherein the outer nosecone comprises an outer nosecone shaft proximal portion extending through the outer balloon catheter lumen, and an outer nosecone shaft distal portion extending between the outer nosecone and the outer nosecone shaft proximal portion, and wherein the outer nosecone shaft distal portion is sized to accommodate the inner balloon therein, in the concealed position of the inner balloon.

Example 45. The delivery assembly of any example herein, particularly any one of examples 1 to 44, wherein the outer nosecone comprises an outer nosecone channel which is sized to allow passage of the inner balloon, in its deflated state, therethrough.

Example 46. The delivery assembly of any example herein, particularly any one of examples 7 to 28, wherein the outer nosecone comprises an outer nosecone channel which is sized to allow passage of the inner nosecone therethrough.

Example 47. The delivery assembly of any example herein, particularly any one of examples 1 to 46, wherein the outer nosecone further comprises an outer nosecone expandable distal portion extending proximally from the distal edge of the outer nosecone.

Example 48. The delivery assembly of any example herein, particularly example 47, wherein the outer nosecone expandable distal portion comprises a plurality of flaps configured to deflect radially outwards.

Example 49. The delivery assembly of any example herein, particularly any one of examples 1 to 48, wherein the delivery apparatus further comprises a push shaft configured to push the guest prosthetic valve from a position proximal to the outer balloon toward the outer balloon.

Example 50. The delivery assembly of any example herein, particularly any one of examples 1 to 49, wherein the host valvular structure is a native valvular structure of native heart valve.

Example 51. The delivery assembly of any example herein, particularly any one of examples 1 to 49, wherein the host valvular structure is a valvular structure of previously implanted prosthetic valve that is implanted within a native heart valve.

Example 52. The delivery assembly of any example herein, particularly any one of examples 1 to 51, wherein the delivery apparatus is sterilized.

Example 53. A method of implanting a guest prosthetic valve within a host valvular structure, the method comprising: advancing a delivery assembly that comprises a delivery apparatus carrying a guest prosthetic valve in a radially compressed state, to a host valvular structure, wherein the delivery apparatus comprises an outer balloon, and an inner balloon axially movable relative to the outer balloon; positioning the inner balloon, in a deflated state thereof, within a host leaflet of the host valvular structure, by axially moving the inner balloon relative to the outer balloon; inflating the inner balloon to form a leaflet opening within the host leaflet to form a leaflet opening within the host leaflet; deflating the inner balloon; positioning the outer balloon in a deflated state thereof, along with the guest prosthetic valve disposed in a compressed state over the outer balloon, inside the host valvular structure; and inflating the outer balloon, so as to radially expand the guest prosthetic valve.

Example 54. The method of any example herein, particularly example 53, wherein the inner balloon is inflatable to a maximum diameter D1.

Example 55. The method of any example herein, particularly example 54, wherein the inflating the outer balloon comprises inflating the outer balloon to a diameter that exceeds D1.

Example 56. The method of any example herein, particularly any one of examples 53 to 55, wherein the delivery apparatus further comprises a perforating member axially movable relative to the inner balloon.

Example 57. The method of any example herein, particularly example 56, wherein the perforating member is axially movable relative to the outer balloon.

Example 58. The method of any example herein, particularly example 56 or 57, further comprising, prior to positioning the inner balloon in the host leaflet, forming, with the perforating member, a pilot puncture within the host leaflet.

Example 59. The method of any example herein, particularly example 58, wherein the positioning the inner balloon in the host leaflet comprises positioning the inner balloon in the pilot puncture.

Example 60. The method of any example herein, particularly example 58 or 59, further comprising, prior to forming the pilot puncture, positioning the perforating member adjacent the host leaflet.

Example 61. The method of any example herein, particularly any one of examples 58 to 60, wherein forming the pilot puncture comprises translating the perforating member in a distal direction relative to the inner balloon and the outer balloon to pierce the host leaflet to form the pilot puncture.

Example 62. The method of any example herein, particularly any one of examples 58 to 61, wherein the perforating member comprises a needle.

Example 63. The method of any example herein, particularly any one of examples 58 to 62, wherein the perforating member comprises a perforating member lumen.

Example 64. The method of any example herein, particularly example 63, wherein forming the pilot puncture comprises perforating the host leaflet by a guidewire extending through the perforating member lumen, followed by piercing the host leaflet by the perforation member, advanced over the guidewire to form the pilot puncture.

Example 65. The method of any example herein, particularly example 64, wherein perforating the host leaflet by the guidewire comprises applying RF energy to a tip of the guidewire.

Example 66. The method of any example herein, particularly any one of examples 58 to 61, wherein the perforating member is a guidewire.

Example 67. The method of any example herein, particularly example 66, wherein forming the pilot puncture comprises applying RF energy to a tip of the guidewire.

Example 68. The method of any example herein, particularly any one of examples 53 to 66, wherein the positioning the outer balloon inside the host valvular structure comprises positioning the outer balloon between host leaflets of the host valvular structure.

Example 69. The method of any example herein, particularly example 68, further comprising, prior to positioning the outer balloon, proximally retracting the deflated inner balloon out of the leaflet opening.

Example 70. The method of any example herein, particularly any one of examples 53 to 66, wherein the positioning the outer balloon inside the host valvular structure comprises positioning the outer balloon inside the leaflet opening.

Example 71. The method of any example herein, particularly example 70, wherein the inflating the outer balloon to radially expand the guest prosthetic valve increases the size of the leaflet opening.

Example 72. The method of any example herein, particularly example 70 or 71, wherein the inflating the outer balloon to radially expand the guest prosthetic valve tears the host leaflet.

Example 73. The method of any example herein, particularly any one of examples 53 to 72, wherein the inflating the outer balloon to radially expand the guest prosthetic valve modifies the host leaflet such that the host leaflet does not obstruct a cell opening of a frame of the guest prosthetic valve.

Example 74. The method of any example herein, particularly any one of examples 53 to 73, wherein the inflating the outer balloon to radially expand the guest prosthetic valve moves the host leaflet to a location upstream of a downstream edge of an outer skirt of the guest prosthetic valve.

Example 75. The method of any example herein, particularly any one of examples 53 to 74, wherein the delivery apparatus further comprises an outer nosecone distal to the outer balloon, and an outer nosecone shaft attached to the outer nosecone.

Example 76. The method of any example herein, particularly example 75, wherein the advancing the delivery assembly to the host valvular structure comprises positioning the outer nosecone in proximity to the host leaflet.

Example 77. The method of any example herein, particularly example 75 or 76, wherein the positioning the outer balloon within the host leaflet comprises advancing the outer nosecone through the leaflet opening.

Example 78. The method of any example herein, particularly any one of examples 75 to 77, wherein the delivery apparatus further comprises an outer balloon catheter, wherein the outer balloon is mounted on the outer balloon catheter and is in fluid communication with the outer balloon catheter.

Example 79. The method of any example herein, particularly example 78, wherein the outer nosecone shaft extends through the outer balloon catheter.

Example 80. The method of any example herein, particularly example 79, wherein the outer nosecone shaft comprises an outer nosecone shaft proximal portion extending through the outer balloon catheter, and an outer nosecone shaft distal portion extending between the outer nosecone and the outer nosecone shaft proximal portion.

Example 81. The method of any example herein, particularly example 80, wherein the outer nosecone shaft distal portion defines an inner diameter that is greater than an inner diameter defined by the outer nosecone shaft proximal portion.

Example 82. The method of any example herein, particularly example 80 or 81, wherein the outer nosecone shaft distal portion is disposed within the outer balloon.

Example 83. The method of any example herein, particularly any one of examples 80 to 82, wherein the advancing the delivery assembly to the host valvular structure comprises retaining the inner balloon within the outer nosecone shaft distal portion.

Example 84. The method of any example herein, particularly any one of examples 80 to 83, wherein the positioning the inner balloon within the host leaflet comprises translating the inner balloon in a distal direction out of the outer nosecone shaft distal portion.

Example 85. The method of any example herein, particularly example 84, wherein translating the inner balloon in a distal direction comprises advancing the inner balloon past the outer nosecone.

Example 86. The method of any example herein, particularly any one of examples 75 to 85, wherein the outer nosecone comprises an outer nosecone channel.

Example 87. The method of any example herein, particularly example 86, wherein the advancing the delivery assembly to the host valvular structure comprises retaining the inner balloon at least partially within the outer nosecone channel.

Example 88. The method of any example herein, particularly example 86 or 87, wherein the positioning the inner balloon within the host leaflet comprises translating the inner balloon in a distal direction through the outer nosecone channel.

Example 89. The method of any example herein, particularly any one of examples 75 to 88, wherein the delivery apparatus further comprises an inner nosecone distal to the inner balloon, and an inner nosecone shaft attached to the inner nosecone.

Example 90. The method of any example herein, particularly example 89, wherein the advancing the delivery assembly to the host valvular structure comprises retaining the inner nosecone proximal to a distal edge of the outer nosecone.

Example 91. The method of any example herein, particularly example 89, wherein the advancing the delivery assembly to the host valvular structure comprises retaining a distal tapering portion of the inner nosecone at least partially distal to the outer nosecone.

Example 92. The method of any example herein, particularly any one of examples 89 to 91, wherein the positioning the inner balloon within the host leaflet comprises advancing the inner nosecone through the host leaflet.

Example 93. The method of any example herein, particularly any one of examples 89 to 92, wherein the outer nosecone comprises an outer nosecone expandable distal portion extending proximally from the distal edge of the outer nosecone.

Example 94. The method of any example herein, particularly example 93, wherein the outer nosecone expandable distal portion comprises a plurality of flaps.

Example 95. The method of any example herein, particularly example 94, wherein the positioning the inner balloon within the host leaflet comprises deflecting the flaps away from each other by advancing the inner nosecone through the inner nosecone.

Example 96. The method of any example herein, particularly any one of examples 53 to 95, further comprising, prior to positioning the outer balloon inside the host valvular structure, distally pushing the guest prosthetic valve, by a push shaft of the delivery apparatus, towards and over the outer balloon.

Example 97. The method of any example herein, particularly example 96, wherein the positioning the outer balloon comprises keeping the push shaft in close proximity to a proximal end of the guest prosthetic valve, so as to provide a counterforce to prevent the guest prosthetic valve from proximally slipping from the outer balloon.

Example 98. The method of any example herein, particularly any one of examples 78 to 85, wherein the inflating the outer balloon comprises providing inflation fluid into the outer balloon via a lumen of the outer balloon catheter.

Example 99. The method of any example herein, particularly any one of examples 89 to 95, wherein the delivery apparatus further comprises an inner balloon catheter, wherein the inner balloon is mounted on the inner balloon catheter and is in fluid communication with the inner balloon catheter.

Example 100. The method of any example herein, particularly example 99, wherein the inner nosecone shaft extends through the inner balloon catheter.

Example 101. The method of any example herein, particularly example 99 or 100, wherein the inflating the inner balloon comprises providing inflation fluid into the inner balloon via a lumen of the inner balloon catheter.

Example 102. The method of any example herein, particularly any one of examples 53 to 101, wherein the host valvular structure is a native valvular structure of native heart valve.

Example 103. The method of any example herein, particularly any one of examples 53 to 101, wherein the host valvular structure is a valvular structure of previously implanted prosthetic valve that is implanted within a native heart valve.

Example 104. The method of any example herein, particularly any one of examples 53 to 103, wherein positioning the inner balloon within the host leaflet comprises positioning the inner balloon within a first host leaflet, wherein inflating the inner balloon within the host leaflet to form the leaflet opening comprises inflating the inner balloon within the first host leaflet to form a first leaflet opening, and wherein, subsequent to deflating the inner balloon, the method further comprises: retracting the delivery apparatus from the first host leaflet; positioning the inner balloon within a second host leaflet; inflating the inner balloon to form a second leaflet opening within the second host leaflet; and deflating the inner balloon.

Example 105. The method of any example herein, particularly example 104, wherein the positioning the outer balloon inside the host valvular structure comprises positioning the outer balloon inside the second leaflet opening.

Example 106. The method of any example herein, particularly any one of examples 53 to 105, further comprising, subsequent to deflating the inner balloon, moving the inner balloon to a concealed position.

Example 107. The method of any example herein, particularly any one of examples 53 to 106, further comprising, subsequent to inflating the outer balloon to radially expand the guest prosthetic valve, deflating the outer balloon and retrieving the delivery apparatus.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A delivery assembly, comprising:
a delivery apparatus comprising:
a handle;
an outer balloon catheter extending from the handle, the outer balloon catheter defining an outer balloon catheter lumen;
an outer balloon mounted on the outer balloon catheter and in fluid communication with the outer balloon catheter lumen, the outer balloon configured to transition between deflated and inflated states thereof;
an outer nosecone distal to the outer balloon;
an outer nosecone shaft attached to the outer nosecone and extending through the outer balloon catheter, the outer nosecone shaft defining an outer nosecone shaft lumen;
an inner balloon catheter extending from the handle through the outer nosecone shaft lumen, the inner balloon catheter defining an inner balloon catheter lumen; and
an inner balloon mounted on the inner balloon catheter and in fluid communication with the inner balloon catheter lumen, the inner balloon configured to transition between deflated and inflated states thereof.

2. The delivery assembly of claim 1, wherein the maximum diameter of the outer balloon in its inflated state is greater than the maximum diameter of the inner balloon in its inflated state.

3. The delivery assembly of claim 1, wherein the inner balloon is movable between a concealed position and an exposed position, wherein the inner balloon is disposed proximal to a distal edge of the outer nosecone when in the concealed position, and wherein the inner balloon is disposed distal to the outer nosecone when in the exposed position.

4. The delivery assembly of claim 3, wherein the balloon is in its deflated state when in the concealed position.

5. The delivery assembly of claim 1, further comprising a guest prosthetic valve comprising a frame movable between a radially compressed and a radially expanded configuration.

6. The delivery assembly of claim 5, wherein, when the guest prosthetic valve is disposed around the outer balloon and positioned within a host valvular structure, inflation of the outer balloon expands the guest prosthetic valve to implant the guest prosthetic valve in the host valvular structure.

7. The delivery assembly of claim 1, further comprising an inner nosecone distal to the inner balloon.

8. The delivery assembly of claim 7, further comprising a perforating member extending through an inner nosecone shaft lumen of an inner nosecone shaft attached to the inner nosecone, the perforating member configured to pierce a host leaflet of a host valvular structure to form a pilot puncture in the host leaflet, when a distal end portion of the perforating member is positioned distal to the outer nosecone and the inner nosecone.

9. The delivery assembly of claim 8, wherein the perforating member comprises a needle.

10. The delivery assembly of claim 3, wherein the outer nosecone comprises an outer nosecone shaft proximal portion extending through the outer balloon catheter lumen, and an outer nosecone shaft distal portion extending between the outer nosecone and the outer nosecone shaft proximal portion, and wherein the outer nosecone shaft distal portion is sized to accommodate the inner balloon therein, in the concealed position of the inner balloon.

11. The delivery assembly of claim 1, wherein the outer nosecone comprises an outer nosecone channel which is sized to allow passage of the inner balloon, in its deflated state, therethrough.

12. The delivery assembly of claim 3, wherein the outer nosecone further comprises an outer nosecone expandable distal portion extending proximally from the distal edge of the outer nosecone.

13. The delivery assembly of claim 12, wherein the outer nosecone expandable distal portion comprises a plurality of flaps configured to deflect radially outwards.

14. A method of implanting a guest prosthetic valve within a host valvular structure, the method comprising:
advancing a delivery assembly that comprises a delivery apparatus carrying a guest prosthetic valve in a radially compressed state, to a host valvular structure, wherein the delivery apparatus comprises an outer balloon, and an inner balloon axially movable relative to the outer balloon,
positioning the inner balloon, in a deflated state thereof, within a host leaflet of the host valvular structure, by axially moving the inner balloon relative to the outer balloon;
inflating the inner balloon to form a leaflet opening within the host leaflet to form a leaflet opening within the host leaflet;
deflating the inner balloon;
positioning the outer balloon in a deflated state thereof, along with the guest prosthetic valve disposed in a compressed state over the outer balloon, inside the host valvular structure; and
inflating the outer balloon, so as to radially expand the guest prosthetic valve.

15. The method of claim 14, wherein the delivery apparatus further comprises a perforating member axially movable relative to the inner balloon.

16. The method of claim 15, further comprising, prior to positioning the inner balloon in the host leaflet, forming, with the perforating member, a pilot puncture within the host leaflet.

17. The method of claim 16, wherein the positioning the inner balloon in the host leaflet comprises positioning the inner balloon in the pilot puncture.

18. The method of claim 16, wherein the forming the pilot puncture comprises translating the perforating member in a distal direction relative to the inner balloon and the outer balloon to pierce the host leaflet to form the pilot puncture.

19. The method of claim 14, wherein the positioning the outer balloon inside the host valvular structure comprises positioning the outer balloon between host leaflets of the host valvular structure.

20. The method of claim 19, further comprising, prior to positioning the outer balloon, proximally retracting the deflated inner balloon out of the leaflet opening.

21. The method of claim 14, wherein the positioning the outer balloon inside the host valvular structure comprises positioning the outer balloon inside the leaflet opening.
